(19) United States
(12) Patent Application Publication
(10) Pub. No.: US 2025/0191682 A1
(43) Pub. Date: Jun. 12, 2025

(54) Integrated Short Read and Long Read Sequencing for Genomic Variant Detection

(71) Applicant: The Broad Institute, Inc., Cambridge, MA (US)

(72) Inventors: Niall Lennon, Cambridge, MA (US); Eric Banks, Newton, MA (US); Fabio Cunial, Cambridge, MA (US); Megan Shand, Somerville, MA (US); Richard Magner, II, Cambridge, MA (US); Sumit Basu, Seattle, WA (US); Timothy Scott Saponas, Woodinville, WA (US); Ron Paulsen, Cambridge, MA (US)

(73) Assignee: The Broad Institute, Inc., Cambridge, MA (US)

(21) Appl. No.: 18/972,558

(22) Filed: Dec. 6, 2024

Related U.S. Application Data
(60) Provisional application No. 63/608,031, filed on Dec. 8, 2023.

(51) Int. Cl. G16B 20/20; G16B 30/10

(52) U.S. Cl. CPC G16B 20/20; G16B 30/10

(57) ABSTRACT

Integrated short read and long read sequencing for genomic variant detection is described. A coverage modulation engine selects a short read sequencing coverage and a long read sequencing coverage for a nucleic acid sequencing event based on a sequencing target of the nucleic acid sequencing event and at least one constraint. A sequencing protocol generator determines a short read sequencing protocol based on the selected short read sequencing coverage and determines a long read sequencing protocol based on the selected long read sequencing coverage. An integrated variant calling module outputs a variant call based on short read sequencing data generated via the short read sequencing protocol and long read sequencing data generated via the long read sequencing protocol.

### RELATED APPLICATION

This application claims priority to U.S. Provisional Patent Application Ser. No. 63/608,031, filed Dec. 8, 2023, entitled “Integrated Short Read and Long Read Sequencing for Genomic Variant Detection,” the entire disclosure of which is hereby incorporated by reference herein in its entirety.

### BACKGROUND

Sequencing technologies have significantly advanced over the years, enabling researchers and clinicians to explore the genetic makeup, including genetic variations, of individuals with increased precision and/or at lower costs. Short read sequencing is cost-effective and provides high accuracy for smaller variants, such as single nucleotide polymorphisms and insertion/deletions. However, short read sequencing is less effective for accurately identifying structural variants and repetitive regions in the genome due to a small read length. On the other hand, long read sequencing technologies provide extended read lengths, making them well-suited for resolving complex genomic regions and structural variants. However, long read sequencing has increased costs in comparison to short read sequencing which can strain research and development budgets.

### SUMMARY

Integrated short read and long read sequencing for genomic variant detection is described. A coverage modulation engine selects a short read sequencing coverage and a long read sequencing coverage for a nucleic acid sequencing event based on a sequencing target of the nucleic acid sequencing event and at least one constraint. A sequencing protocol generator determines a short read sequencing protocol based on the selected short read sequencing coverage and determines a long read sequencing protocol based on the selected long read sequencing coverage. An integrated variant calling module outputs a variant call based on short read sequencing data generated via the short read sequencing protocol and long read sequencing data generated via the long read sequencing protocol.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ integrated short read and long read sequencing for genomic variant detection.

FIG. 2 depicts an example of an implementation of the protocol generation module of FIG. 1 in greater detail.

FIG. 3 depicts an example of an implementation of the coverage modulation engine of FIG. 1 in greater detail.

FIG. 4 depicts a simplified illustrative example of selecting short read and long read sequencing coverage based on modulation inputs.

FIG. 5 depicts a workflow in an example implementation of variant calling by the sequencing integrator of FIG. 1.

FIG. 6 shows an example of an alignment in a first example of integrating short read and long read sequencing for genomic variant detection.

FIG. 7 shows an example of an alignment in a second example of integrating short read and long read sequencing for genomic variant detection.

FIG. 8 illustrates an example of integrating short read and long read sequencing to detect phasing.

FIG. 9 depicts an example procedure in which sequencing coverage is modulated for a sequencing event.

FIG. 10 depicts an example procedure in which a combination of short read sequencing data and long read sequencing data is used for variant calling.

FIG. 11 illustrates an example system including various components of an example device that can be implemented as any type of computing device as described and/or utilized with reference to FIGS. 1-10 to implement embodiments of the techniques described herein.

FIG. 12 shows a first example alignment of hybrid short read and long read sequencing data.

FIG. 13 shows a second example alignment of hybrid short read and long read sequencing data.

FIG. 14 shows a third example alignment of hybrid short read and long read sequencing data.

FIG. 15 shows an example classification of new single nucleotide polymorphisms identified using a hybrid readset of short read and long read sequencing data.

FIG. 16 shows an example of structural variant discovery with long reads versus short reads.

FIG. 17 shows an example of insertion structural variant discovery with long reads versus short reads.

FIG. 18 shows an example of deletion structural variant discovery with long reads versus short reads.

FIG. 19 shows an example of structural variant discovery for structural variants of length 100-250 base pairs with long reads versus short reads.

FIG. 20 shows an example of structural variant discovery for structural variants of length 2500-10000 base pairs with long reads versus short reads.

FIG. 21 shows an example of genotype-aware structural variant discovery with long reads alone, short reads alone, or a hybrid method.

### DETAILED DESCRIPTION

### Overview

Genome interpretation refers to a systematic analysis of the information encoded in an individual's genetic material, particularly the individual's deoxyribonucleic acid (DNA) sequence. This process involves identifying and annotating genetic variations, understanding their potential functional impacts, and correlating these findings with relevant biological and clinical knowledge. Genome interpretation aims to unveil the genetic basis of traits, diseases, and individual responses to therapeutic interventions, which contributes to the development of medicines (e.g., personalized medicines).

For example, bioinformatics techniques can be applied to process and analyze genomic data to determine qualitative and quantitative information about that data. Genomic data is typically generated via DNA sequencing techniques. For instance, manual or automated DNA sequencing techniques may be used to determine a sequence of nucleotide bases in a DNA sample obtained from a subject. However, DNA sequencing techniques do not produce full-length chromosomal sequences of the DNA sample. Rather, sequence fragments are produced without any indication of their location in the genome. One DNA sequencing technique referred to as short read sequencing produces sequence fragments that typically range from approximately 50 base pairs to approximately 500 base pairs, whereas another DNA sequencing technique called long read sequencing produces sequence fragments that are typically greater than 10,000 base pairs, such as in a range from 10,000 to 100,000 base pairs or more (e.g., two million base pairs).

Because the human genome is approximately 3 billion base pairs, even sequence fragments generated by long read sequencing are a fraction of the human genome. Therefore, in order to generate full-length genomic sequences or portions thereof, these sequence fragments are mapped (e.g., aligned) with a reference genomic sequence (e.g., “reference genome”) and merged based on their positioning and overlap with respect to each other in a process referred to as alignment. This results in a consensus sequence, e.g., the genomic sequence of the sample. Sequencing coverage refers to the average number of times a given nucleotide in a nucleic acid sample is read or sequenced, and higher coverage generally results in a more accurate consensus sequence.

Moreover, the genomic sequence of the sample may be compared to the reference genomic sequence to determine how the genomic sequence of the sample varies from that of the reference in a process referred to as variant calling. Variant calling includes identifying and characterizing genetic variations, or variants, in the genomic sequence of the sample in comparison to the reference genomic sequence. These variants can include short variants, such as single nucleotide polymorphisms (SNPs, where one nucleotide is changed to another at a specific position in the genome), insertions (e.g., the addition of less than 50 nucleotides at a particular location in the genome), and deletions (e.g., the removal of less than 50 nucleotides at a specific position in the genome). These variants can also include larger structural variations, such as copy number variants (CNVs, where a segment of DNA ranging from kilobases to megabases in size is duplicated or deleted), inversions (e.g., a segment of DNA is reversed in orientation), larger insertions or deletions (e.g., INDELs) involving larger segments (e.g., more than 50 nucleotides), and translocations (e.g., where a segment of DNA from one location to another, often involving the exchange of genetic material between non-homologous chromosomes).

Existing methods for variant calling often rely exclusively on either short read or long read sequencing technologies. For example, short read sequencing is effective for identifying short variants and has a lower cost than long read sequencing, whereas long read sequencing is more effective for identifying structural variants but may be quite costly. This hampers the ability to comprehensively identify genetic variants, especially in regions with inherent complexities. Accurate identification of variants enhances an understanding of disease etiology, guides personalized medicine, and/or enhances population genetics information.

To overcome these problems, integrated short read and long read sequencing for genomic variant detection is disclosed. In accordance with the techniques described herein, short read sequencing data is augmented with long read sequencing data. The augmentation can provide cost- and/or resource-efficient variant detection of both short variants and structural variants. By way of example, short read coverage may be reduced compared with traditional short read sequencing techniques due to augmentation by the long read sequencing data while enhancing sensitivity and/or precision of the variant detection. Additionally, or alternatively, long read coverage may be reduced compared to traditional long read sequencing techniques due to the inclusion of short read data while enhancing the sensitivity and/or precision of the variant detection. By synergistically leveraging the high accuracy of short reads for small variants and the ability of long reads to traverse complex genomic regions and detect larger structural variants, highly accurate and/or sensitive variant calling is achieved.

In one or more implementations, the short read coverage and the long read coverage are modulated to provide customizable sequencing outcomes with respect to variant calling sensitivity and precision as well as experimental cost. For instance, the long read sequencing may be targeted to specific genomic region(s), and so even though the long read coverage is reduced on average across the genome, the long read coverage may remain relatively high at the targeted genomic region(s). Additionally, or alternatively, the long read sequencing data may be used to augment a reference genome so that short read sequencing data is able to be aligned to the reference genome in areas that are otherwise difficult to map, such as highly repetitive regions or regions having other structural variants, due to deviations from the reference genome. As such, where desired, the short read coverage and the long read coverage may both be reduced without losing the genomic information typically determined through higher coverage techniques.

In some aspects, the techniques described herein relate to a system including: a coverage modulation engine to select a short read sequencing coverage and a long read sequencing coverage for a nucleic acid sequencing event based on a sequencing target of the nucleic acid sequencing event and at least one constraint; a sequencing protocol generator to determine a short read sequencing protocol based on the selected short read sequencing coverage and determine a long read sequencing protocol based on the selected long read sequencing coverage; and an integrated variant calling module to output a variant call based on short read sequencing data generated via the short read sequencing protocol and long read sequencing data generated via the long read sequencing protocol.

In some aspects, the techniques described herein relate to a system, wherein the coverage modulation engine generates and stores relationships between the at least one constraint and sequencing coverages for a plurality of different combinations of short read and long read sequencing coverages and based on reference short read sequencing data and reference long read sequencing data obtained for a reference sample.

In some aspects, the techniques described herein relate to a system, wherein the coverage modulation engine selects the short read sequencing coverage and the long read sequencing coverage based on the relationships and based on a multi-objective algorithm.

In some aspects, the techniques described herein relate to a system, further including an alignment module to: align the short read sequencing data and the long read sequencing data with respect to one or more reference sequences and with respect to each other; and generate a sequence of a sample evaluated by the nucleic acid sequencing event based on an order of nucleotide bases specified by the aligned short read sequencing data and the aligned long read sequencing data.

In some aspects, the techniques described herein relate to a system, wherein the variant call output by the integrated variant calling module is further based on one or more differences between the sequence of the sample and the one or more reference sequences.

In some aspects, the techniques described herein relate to a system, wherein the at least one constraint includes one or more of a sensitivity for identifying at least one type of variant in at least a portion of a reference sequence, a precision for identifying the at least one type of variant in at least the portion of the reference sequence, and an accuracy of identifying nucleotide bases in the reference sequence.

In some aspects, the techniques described herein relate to a system, wherein a sensitivity of the variant call and a precision of the variant call are increased compared to performing the variant call using the short read sequencing data without the long read sequencing data and compared to performing the variant call using the long read sequencing data without the short read sequencing data.

In some aspects, the techniques described herein relate to a system, wherein the variant call identifies at least one of a single nucleotide polymorphism, an insertion, a deletion, a copy number variant, a duplication, an inversion, a replacement, or a translocation in a sample sequenced by the nucleic acid sequencing event relative to a reference sequence, and wherein the variant call further includes variant phasing information.

In some aspects, the techniques described herein relate to a method including: receiving a selection of at least one target of a genomic sequencing event and at least one constraint of the genomic sequencing event; selecting a short read sequencing coverage and a long read sequencing coverage for the genomic sequencing event based on the at least one target and the at least one constraint; generating a short read sequencing protocol based on the short read sequencing coverage; and generating a long read sequencing protocol based on the long read sequencing coverage.

In some aspects, the techniques described herein relate to a method, wherein the at least one constraint includes a performance constraint defining a targeted performance metric of the genomic sequencing event and a resource constraint of the genomic sequencing event.

In some aspects, the techniques described herein relate to a method, wherein the targeted performance metric is one or more performance metrics selected from a group including an accuracy metric, a sensitivity metric, and a precision metric.

In some aspects, the techniques described herein relate to a method, wherein selecting the short read sequencing coverage and the long read sequencing coverage for the genomic sequencing event based on the at least one target and the at least one constraint includes: referencing a relationship library storing a plurality of relationships between the at least one constraint and sequencing coverages for a plurality of different combinations of short read and long read sequencing coverages obtained for a reference sample and a plurality of different targets.

In some aspects, the techniques described herein relate to a method, further including sequencing a nucleic acid sample using the long read sequencing protocol and the short read sequencing protocol.

In some aspects, the techniques described herein relate to a method including: receiving short read sequencing data generated for a biological sample via a short read sequencing protocol having a first sequencing coverage, the short read sequencing data including sequences of nucleotide bases for a plurality of short nucleotide fragments; receiving long read sequencing data generated for the biological sample via a long read sequencing protocol having a second sequencing coverage, the long read sequencing data including sequences of the nucleotide bases for a plurality of long nucleotide fragments, wherein the first sequencing coverage and the second sequencing coverage are selected based on at least one constraint; generating at least one alignment of the short read sequencing data and the long read sequencing data with a reference genome; and outputting a variant call based on the at least one alignment relative to the reference genome.

In some aspects, the techniques described herein relate to a method, wherein the first sequencing coverage and the second sequencing coverage are selected by a coverage modulation engine using a multi-objective algorithm and based on the at least one constraint, and wherein the at least one constraint includes one or more of a sensitivity for identifying at least one type of variant in at least a portion of a genomic sequence, a precision for identifying the at least one type of variant in at least the portion of the genomic sequence, an accuracy of identifying the nucleotide bases in at least the portion of the genomic sequence, and a resource constraint for generating the genomic sequence of the biological sample.

In some aspects, the techniques described herein relate to a method, wherein the first sequencing coverage and the second sequencing coverage are further selected by the coverage modulation engine based on a sequencing target.

In some aspects, the techniques described herein relate to a method, wherein outputting the variant call based on the at least one alignment relative to the reference genome includes: after aligning the long read sequencing data, fragmenting the long read sequencing data into aligned synthetic short reads; generating a hybrid alignment by combining the aligned synthetic short reads with a short read alignment of the short read sequencing data; and outputting the variant call based on the hybrid alignment.

In some aspects, the techniques described herein relate to a method, wherein generating the at least one alignment of the short read sequencing data and the long read sequencing data with the reference genome includes: generating an augmented reference genome by mapping the long read sequencing data to the reference genome; and mapping the short read sequencing data to the augmented reference genome.

In some aspects, the techniques described herein relate to a method, wherein the first sequencing coverage includes an average number of reads at a specific location in a genome that is less than thirty, and wherein the second sequencing coverage is less than the first sequencing coverage.

In some aspects, the techniques described herein relate to a method, further including: identifying phasing of variants output in the variant call, the phasing indicating which of the variants occur on a same copy of a chromosome.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

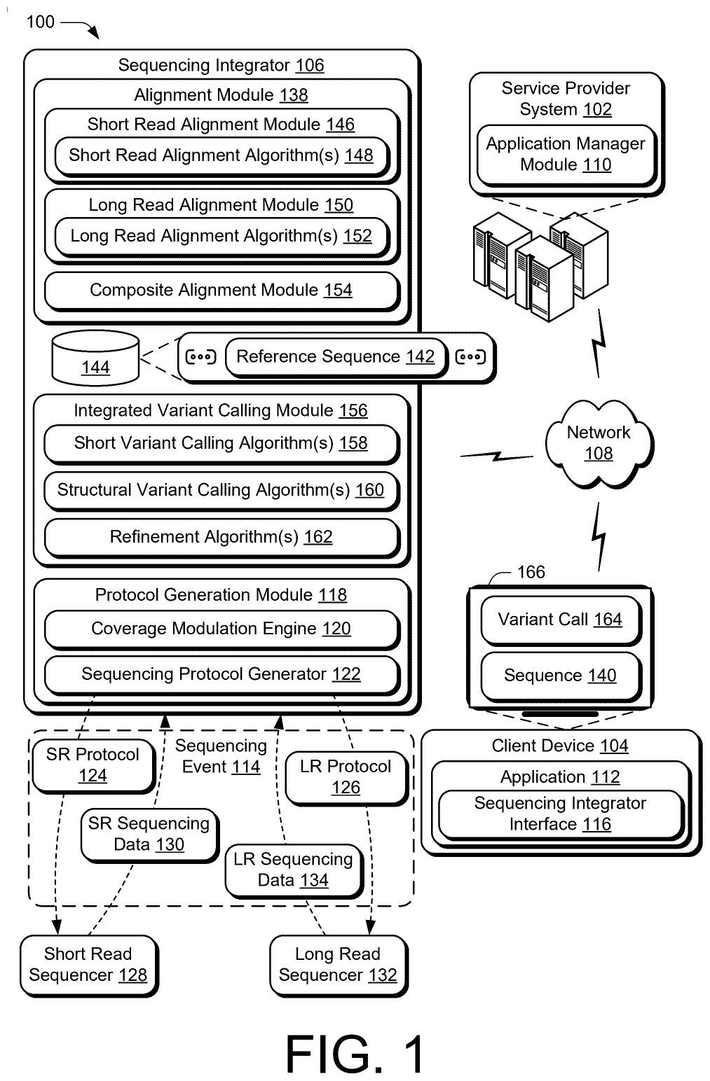

FIG. 1 is an illustration of an environment 100 in an example implementation that is operable to employ integrated short read and long read sequencing for genomic variant detection as described herein. The illustrated environment 100 includes a service provider system 102, a client device 104, and a sequencing integrator 106 that are communicatively coupled, one to another, via a network 108. Although functionality of the sequencing integrator 106 is illustrated as separate from the service provider system 102, this functionality may be incorporated as part of the service provider system 102, further divided among other entities, and so forth. Additionally, or alternatively, an entirety of or portions of the functionality of the sequencing integrator 106 may be incorporated as part of the client device 104.

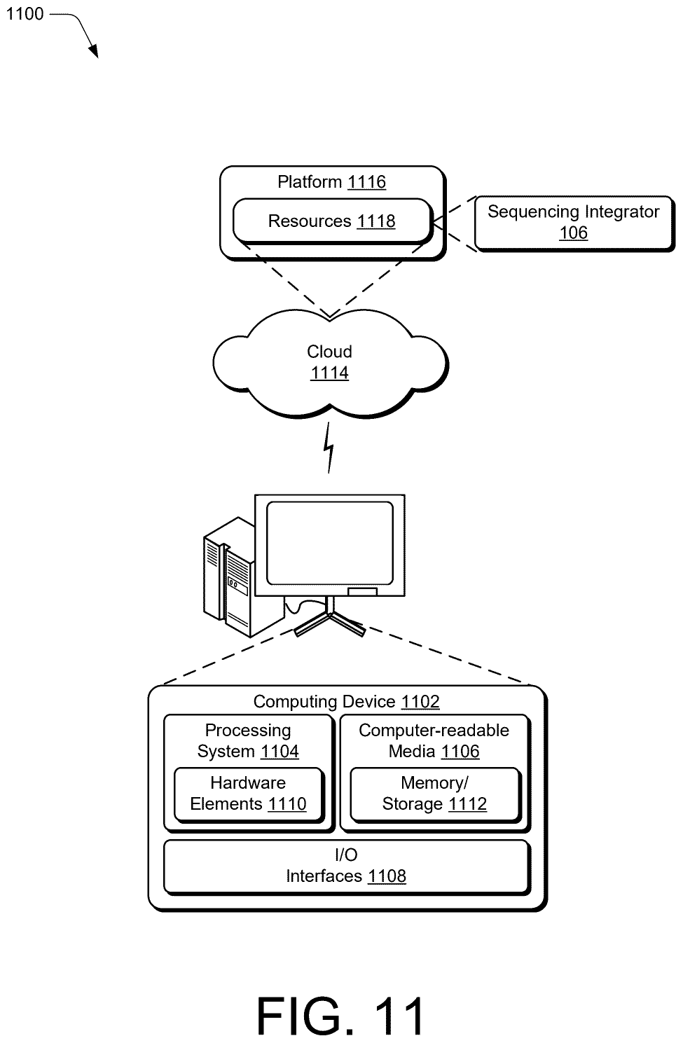

Computing devices that are usable to implement the service provider system 102, the client device 104, and the sequencing integrator 106 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 11.

The service provider system 102 is illustrated as including an application manager module 110 that is representative of functionality to provide access to the sequencing integrator 106 to a user of the client device 104 via the network 108. The application manager module 110, for instance, may expose content or functionality of the sequencing integrator 106 that is accessible via the network 108 by an application 112 of the client device 104. The application 112 may be configured as a network-enabled application, a browser, a native application, and so on, that exchanges data with the service provider system 102 via the network 108. The data can be employed by the application 112 to enable the user of the client device 104 to communicate with the service provider system 102, such as to receive application updates and features when the service provider system 102 provides functionality to manage the application 112.

In the context of the described techniques, the application 112 includes functionality to generate sequencing protocols for a sequencing event 114 as well as to analyze data generated by the sequencing event 114. In the illustrated example, the application 112 includes a sequencing integrator interface 116 that is implemented at least partially in hardware of the client device 104 for facilitating communication between the client device 104 and the sequencing integrator 106. By way of example, the sequencing integrator interface 116 includes functionality to receive inputs to the sequencing integrator 106 from the client device 104 (e.g., from a user of the client device 104) and output information, data, and so forth from the sequencing integrator 106 to the client device 104, as will be further elaborated herein.

The sequencing event 114 is an occurrence of determining an order of nucleotides (e.g., adenine, thymine or uracil, cytosine, and guanine) in a sample of nucleic acids, such as derived from a biological sample. The order of nucleotides is referred to herein as a “sequence.” The nucleotides are also referred to as “bases.” Although the sequencing event 114 will be described herein with respect to deoxyribonucleic acid (DNA) sequencing, and more particularly genomic sequencing, it is to be appreciated that the techniques described herein may be adapted for sequencing other types of nucleic acids, such as ribonucleic acid (RNA). By way of example, sequencing can be performed using genomic DNA, complementary DNA (cDNA) derived from RNA transcripts, or RNA as a template.

Various sequencing techniques are available for producing sequencing data that is usable to determine the order of nucleotides in a sample. Short read sequencing techniques produce sequence fragments that typically range from approximately 10 bases to approximately 1000 bases and more typically approximately 50 bases to approximately 500 bases. Sequence fragments produced via short read sequencing techniques are also referred to herein as “short reads.” Long read sequencing techniques produce sequence fragments that typically range from 2000 bases to 1,000,000 bases and more typically from 5000 bases to 500,000 bases in length. Sequence fragments produced via long read sequencing techniques are also referred to herein as “long reads.” In general, short read sequencing techniques are higher throughput, which enables sequencing a plurality of nucleic acid fragments (e.g., DNA fragments) in parallel. At least in part due to this high-throughput, short read sequencing has a lower cost per base compared with long read sequencing.

In accordance with the techniques described herein, the sequencing integrator 106 is representative of functionality to integrate short read sequencing technologies and long read sequencing technologies for effective and cost-effective variant calling for the sequencing event 114. “Variant calling” refers to the identification and/or characterization of genetic variations, or “variants,” in a sequence determined for a sample (e.g., an individual's genome) when compared to a reference sequence (e.g., a reference genome). These variants may include short variants, such as single nucleotide polymorphisms (SNPs, where one nucleotide is changed to another at a specific position in the genome), insertions (e.g., the addition of less than 50 nucleotides at a particular location in the genome), and deletions (e.g., the removal of less than 50 nucleotide at a specific position in the genome). Insertions and deletions may be collectively referred to as “INDELs.” Additionally, or alternatively, the variants may include larger, structural variations, such as copy number variants (CNVs, where a segment of DNA ranging from kilobases to megabases in size is duplicated or deleted), inversions (e.g., where a segment of DNA is reversed in orientation), INDELs involving larger segments (e.g., more than 50 nucleotides), translocations (e.g., where a segment of DNA is moved from one location to another, often involving the exchange of genetic material between non-homologous chromosomes), and replacements (e.g., where a segment of DNA is replaced or substituted by another, which may include additional changes such as insertions, duplications, or other rearrangements).

Moreover, the structural variants may be “complex events,” which include a combination of structural changes (e.g., CNVs, large INDELs, duplications, inversions, and translocations) within a specific region in the genome. Examples of complex event structural variants include nested insertions (e.g., where one DNA fragment is inserted into another, with the inserted fragment itself containing additional structural changes), inverted duplications with deletions (e.g., wherein a DNA segment is duplicated one or more times at a region of the genome, with at least one duplicate copy inverted and/or including a deletion), inversions with translocations (e.g., where a DNA segment is both inverted and relocated to a different location in the genome), and complex chromosomal rearrangements (e.g., a combination of translocations, inversions, deletions, and/or duplications at a specific genomic region). Additionally, or alternatively, the structural variants may be included in “composite events” where individual structural variants occur close to one another in a specific genomic region. Non-limiting examples of composite events include deletion-insertion composites, inversion-translocation composites, and duplication-deletion composites.

Short read sequencing is associated with high sequencing accuracy, with low error rates per base, making short read sequencing effective for detecting short variants (e.g., SNPs and short INDELs). Because long reads can span repetitive and complex genomic regions, long read sequencing is typically more effective for providing more contiguous and accurate genome assembly as well as detecting structural variants (e.g., CNVs, large INDELs, duplications, inversions, translocations, and/or combinations thereof). As such, the sequencing integrator 106 described herein combines short read sequencing and long read sequencing in a customizable manner to increase the amount of genomic information gained per dollar spent on sequencing.

In the example of the environment 100 shown in FIG. 1, the sequencing integrator 106 includes a protocol generation module 118. Broadly, the protocol generation module 118 is representative of functionality for selecting and/or adjusting a sequencing coverage (e.g., depth) of the sequencing event 114 and generating sequencing protocols accordingly. “Sequencing coverage” or “sequencing depth” refers to the average number of times a given nucleotide in a nucleic acid sample is read or sequenced during the sequencing event 114 (e.g., a sequencing experiment). In general, higher coverage results in more accurate variant calling and the detection of rare variants but comes at the cost of increased sequencing resources. Moreover, increasing sequencing coverage has diminishing returns; extremely high coverage may not provide significant additional benefits while being more resource intensive. In contrast, decreasing sequence coverage enables more samples to be evaluated for a given amount of data produced by a sequencer, which enables multiplexing and reduces sequencing costs. Sequencing coverage is typically given as a multiple of an average coverage value, such as “30×” to denote that each base in a nucleic acid sample has been sequenced thirty times on average.

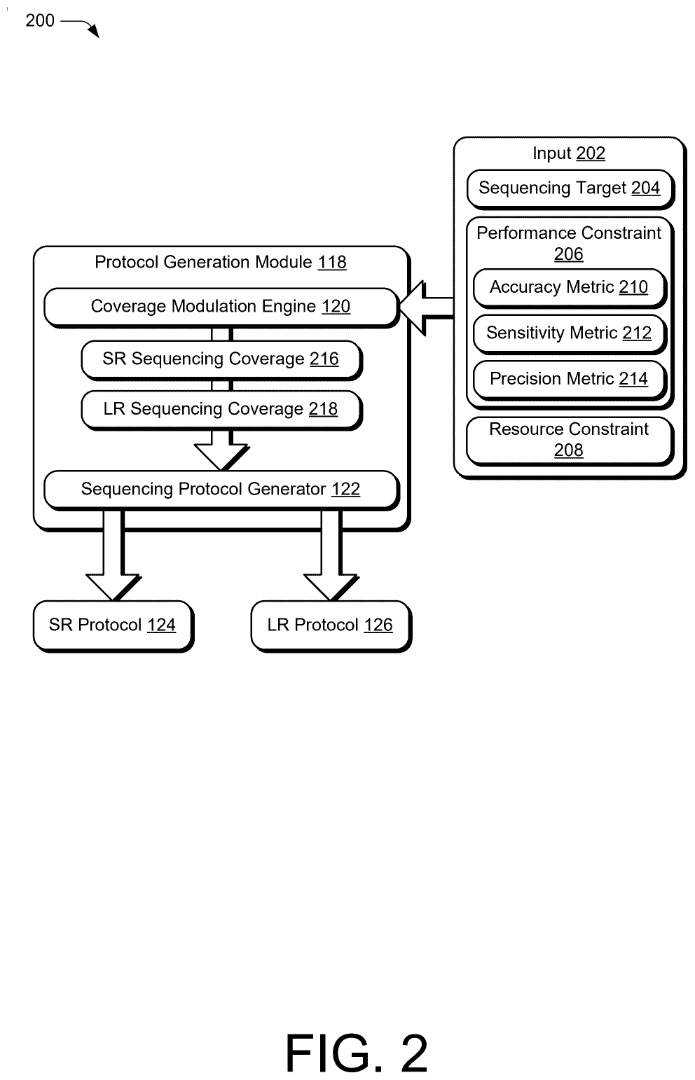

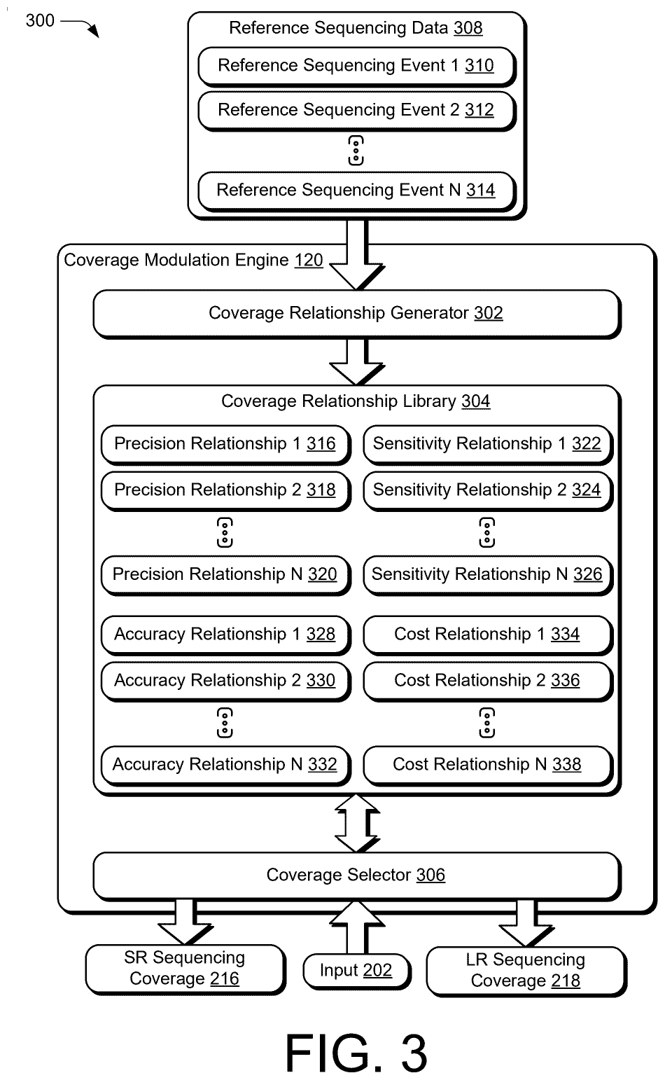

Accordingly, in at least one implementation, the protocol generation module 118 includes a coverage modulation engine 120 and a sequencing protocol generator 122. As will be elaborated below, e.g., with respect to FIGS. 2 and 3, the coverage modulation engine 120 is configured to receive at least one input regarding the sequencing event 114 (such as a sequencing target, a performance constraint, and/or a cost constraint) and output a combination of short read sequencing coverage and long read sequencing coverage based on the at least one input. By way of example, the coverage modulation engine 120 generates and uses a plurality of defined relationships between sequencing coverage and sequencing performance (e.g., sensitivity, accuracy, and/or precision) for a plurality of different sequencing targets (e.g., a type of variant targeted, a genomic region targeted, and/or a desired sequencing technology) as well as with respect to sequencing cost (e.g., monetary and/or resource cost). These defined relationships enable both a short read sequencing coverage and a long read sequencing coverage to be modulated according to a target and/or goal of the sequencing event 114. Using the defined relationships, for instance, the coverage modulation engine 120 selects a combination of short read coverage and long read coverage that is expected to provide a desired sequencing performance for a specific sequencing target for a lowest cost.

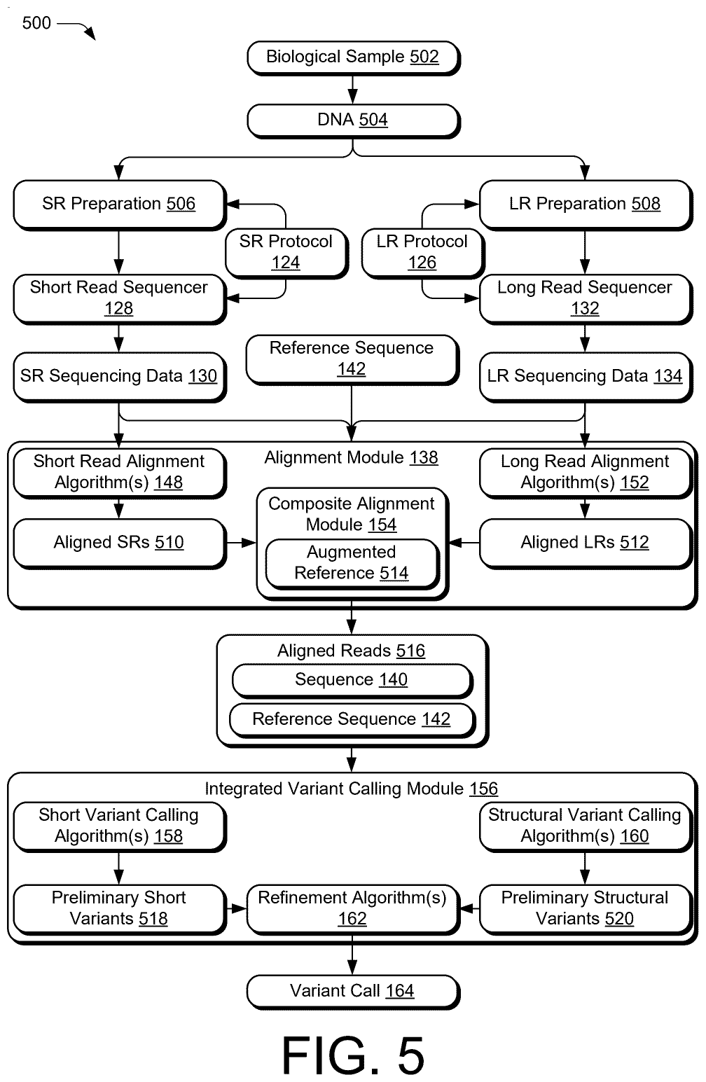

In at least one implementation, the sequencing protocol generator 122 is configured to generate and output a short read protocol 124 (abbreviated in the figures as “SR protocol 124”) based on the selected short read coverage and a long read protocol 126 (abbreviated in the figures as “LR protocol 126”) based on the selected long read coverage, as selected or otherwise determined by the coverage modulation engine 120. In one or more implementations, the short read protocol 124 and/or the long read protocol 126 includes instructions executable by a corresponding sequencing platform to generate sequencing data. Additionally, or alternatively, the short read protocol 124 and/or the long read protocol 126 includes instructions for an experimental setup performed by a user, such as how to multiplex a sequencing run. Additional details regarding sample preparation will be described herein with respect to FIG. 5. For example, in at least one implementation, the short read protocol 124 and the long read protocol 126 include a strategy for linking samples prepared for long read sequencing to those prepared for short read sequencing, such as through tagging in a manner that maintains correspondence between the two sequencing techniques. In at least one variation, however, the samples prepared for short read sequencing are not directly linked to the samples prepared for long read sequencing.

The short read protocol 124 is usable by a short read sequencer 128 to perform short read sequencing and generate short read sequencing data 130 (abbreviated in the figures as “SR sequencing data”). The short read sequencing data 130 comprise a large collection of short nucleic acid sequences, or short reads, that represent small fragments of the original nucleic acid sample that was sequenced. For instance, a short read includes an ordered combination of nucleotides with a length typically ranging from tens to hundreds of bases, such as discussed above. The short read sequencer 128 employs a short read sequencing technique, such as next-generation sequencing or Sanger sequencing, to determine the order of nucleotides in each short read and generate the short read sequencing data 130. By way of example, the short read sequencer 128 may employ a paired-end sequencing approach where two short reads are obtained from opposite ends of a DNA fragment, which provides additional information about relationships and distances between the sequenced fragments. Thus, in at least one implementation, the short read sequencing data 130 includes short read pairs (e.g., a “forward” read and a “reverse” read).

Similarly, the long read protocol 126 is usable by a long read sequencer 132 to perform long read sequencing and generate long read sequencing data 134. The long read sequencing data 134 comprise a large collection of longer nucleic acid sequences, or long reads, that represent larger fragments (e.g., in comparison to the short reads) of the original nucleic acid sample that was sequenced. For instance, a long read includes an ordered combination of nucleotides with a length typically ranging from thousands to hundreds of thousands of bases, such as discussed above. In one or more implementations, the long read sequencing data 134 further include methylation data. The long read sequencer 132 employs a long read sequencing technique, such as single molecule real-time sequencing or nanopore sequencing, to determine the order of nucleotides in each long read and generate the long read sequencing data 134.

In at least one implementation, the long read sequencing data 134 are generated by multiple different long read sequencers and/or using different types of long read sequencing techniques. By way of example, a first portion of the long read sequencing data 134 may be generated by a first long read sequencer and/or using a first long read sequencing technique, and a second, remaining portion of the long read sequencing data 134 may be generated by a second long read sequencer and/or using a second long read sequencing technique. Doing so may enable mixing of different sequencing techniques that provide different advantages, such as mixing targeted long read sequencing with ultralong sequencing.

In one or more implementations, the sequencing integrator 106 further includes an alignment module 138. The alignment module 138 is representative of functionality for determining a sequence 140 (e.g., consensus sequence) of the sample investigated via the sequencing event 114. Read alignment, also referred to simply as “alignment,” involves mapping nucleic acid segments to locations in the genome. The alignment module 138 is configured to receive the short read sequencing data 130 and the long read sequencing data 134 and align (e.g., map) the short read sequencing data 130 and the long read sequencing data 134 to a reference sequence 142. In at least one implementation, the short read sequencing data 130 and the long read sequencing data 134 are provided in a text-based file format, such as FASTQ files that store both nucleotide sequence information and quality scores for the bases in a sequencing read. In variations, the short read sequencing data 130 and the long read sequencing data 134 comprise another type of file format.

It is contemplated that the reference sequence 142 can be selected from a variety of nucleic acid sequences against which a sequence of a sample can be compared for determining variants of the sequence of the sample. The reference sequence 142 is a reference genome or a portion thereof. In one or more implementations, the sequencing integrator 106 includes or otherwise accesses a storage device 144 storing the reference sequence 142. The storage device 144 may store one or more other reference sequences in addition to the reference sequence 142, as indicated by ellipses in FIG. 1. By way of example, different reference sequences may correspond to different sample types or may come from a pangenome, which includes several high-quality, curated assemblies of individual genomes that may be represented jointly as a graph. As such, the reference sequence 142 is selected based on its similarity to the sample evaluated via the sequencing event 114, at least in some implementations. Moreover, the reference sequence 142 may include a combination of more than one individual reference sequences.

In at least one implementation, the alignment module 138 includes a short read alignment module 146 configured to map the short read sequencing data 130 to the reference sequence 142 via one or more short read alignment algorithms 148, a long read alignment module 150 configured to map the long read sequencing data 134 to the reference sequence 142 via one or more long read alignment algorithms 152, and a composite alignment module 154 configured to align the short read sequencing data 130 and the long read sequencing data 134 with respect to each other. In a non-limiting example further described herein, the composite alignment module 154 is configured to generate an augmented reference sequence (e.g., a personalized graph-based reference) using the long read sequencing data 134 and the long read alignment module 150. The short read alignment module 146 may then use the augmented reference to align the short read sequencing data 130. For instance, accurate alignment of the short read sequencing data 130 to the reference sequence 142 can be hindered by regions of the genome having repetitive sequences, such as short tandem repeats (STRs) or other structural variants that are not accounted for in the reference sequence 142. Because the long read sequencing data 134 has extended reads in comparison to the long read sequencing data 134, the long read sequencing data 134 offers the ability to resolve repetitive regions or identify the locations of other structural variants in the sequence 140.

The sequencing integrator 106 further includes an integrated variant calling module 156 representative of functionality for determining genomic differences between the sequence 140 and the reference sequence 142. By way of example, after the short reads and the long reads are aligned to the reference sequence 142, variants are detected by identifying positions where the sequence 140 (or an alignment indicating the sequence 140) differs from the reference sequence 142. In at least one implementation, the integrated variant calling module 156 includes one or more short variant calling algorithms 158, one or more structural variant calling algorithms 160, and/or one or more refinement algorithms 162. The one or more short variant calling algorithms 158, the one or more structural variant calling algorithms 160, and the one or more refinement algorithms 162 are configured to be used alone or in any combination to generate a variant call 164. The variant call 164 includes an indication of one or more variants, including small variant(s) and/or structural variant(s), that are determined to be present in the sequence 140 compared to the reference sequence 142.

In at least one implementation, the one or more short variant calling algorithms 158, the one or more structural variant calling algorithms 160, and/or the one or more refinement algorithms 162 employ statistical and/or computational methods to distinguish true genetic variants from sequencing errors and artifacts. The one or more refinement algorithms 162, for instance, include functionality to adjust and/or adapt the one or more short variant calling algorithms 158 and/or the one or more structural variant calling algorithms 160 based on the short read coverage and/or the long read coverage selected by the coverage modulation engine 120. By way of example, the short read coverage may be reduced compared to short read sequencing (e.g., traditional short read sequencing) that is not augmented by long read sequencing, and so the one or more refinement algorithms 162 may tune the one or more short variant calling algorithms 158 based on the reduced coverage and further based on the information provided by the long read sequencing data 134. Similar tuning may be performed on the structural variant calling algorithms 160. For instance, traditional short read sequencing may be performed at 30× short read coverage, and so the one or more refinement algorithms 162 may tune the one or more short variant calling algorithms 158 to make short variant calls using less than 30× short read coverage while maintaining a targeted (or adequate) sensitivity and/or precision. Similarly, traditional long read sequencing may be performed at 30× long read coverage, and so the one or more refinement algorithms 162 may tune the one or more structural variant calling algorithms 160 to make structural variant calls using less than 30× long read coverage while maintaining a targeted (or adequate) sensitivity and/or precision.

By way of example, the one or more refinement algorithms 162 may be deep learning algorithms configured to train the one or more short variant calling algorithms 158 and/or the one or more refinement algorithms 162 based on the short read coverage and/or the long read coverage selected by the coverage modulation engine 120. For instance, the one or more refinement algorithms 162 may use deep learning techniques to train the one or more short variant calling algorithms 158 with short read data acquired at the selected short read coverage for a reference sample having a known sequence and/or long read data acquired at the selected long read coverage for the reference sample. Similarly, the one or more refinement algorithms 162 may use deep learning techniques to train the one or more structural variant calling algorithms 160 using deep learning techniques and the short read data acquired at the selected short read coverage for the reference sample and/or the long read data acquired at the selected long read coverage for the reference sample. By way of example, the one or more structural variant calling algorithms 160 may be tuned (e.g., by the one or more refinement algorithms 162) to make structural variant calls using a small number of reads of evidence (e.g., two or less), rather than the greater number of reads of evidence (e.g., greater than two) that is typically utilized, such as discussed above.

In one or more implementations, the one or more short variant calling algorithms 158 may be trained to identify short variants based on the long read sequencing data 134 in addition to or as an alternative to using the short read sequencing data 130. Similarly, the one or more structural variant calling algorithms 160 may be trained to identify structural variants based on the short read sequencing data 130 in addition to or as an alternative to using the long read sequencing data 134. As such, the short read sequencing data 130 and the long read sequencing data 134 may be utilized in various ways by the variant calling algorithms in accordance with the techniques described herein.

In at least one implementation, the training produces a joint model from the one or more short variant calling algorithms 158 and the one or more structural variant calling algorithms 160 for combined short variant and structural variant calling that is specific for the short read coverage and the long read coverage used in the sequencing event 114. In at least one variation, however, rather than generating a joint model, the tuned one or more short variant calling algorithms 158 and the tuned one or more structural variant calling algorithms 160 are utilized in separate variant calling pipelines, and the one or more refinement algorithms 162 additionally or alternatively include functionality for taking a union of these separate variant calling pipelines. For instance, the one or more refinement algorithms 162 include filtering and/or genotyping algorithms that are tuned based on the selected short read and long read coverages in order to maintain a desired or adequate precision and/or sensitivity of the variant calling.

In one or more implementations, in addition to or as an alternative to the processes described above, structural variants determined by the one or more structural variant calling algorithms 160 from the long read sequencing data 134 may be filtered (e.g., to increase precision) by using a set of features that come from both the long and the short read sequencing data 130. As used herein, a set of features may refer to a collection of characteristics, attributes, or data points derived from long read sequencing data and/or short read sequencing data. These features may include, but are not limited to, read depth, mapping quality, base quality scores, alignment scores, sequence context, base pairs, and variant allele frequencies. The set of features may be used to evaluate and filter structural variants, which may improve the precision of variant calls by leveraging complementary information from both long read and short read sequencing technologies. By way of example, the structural variants and the reference sequence 142 may be used to generate a variation graph (or graph-based augmented reference), and the short read sequencing data 130 may be aligned to the variation graph. Structural variants at locations on the variation graph that are supported by at least a threshold number of short read alignments are considered to be validated. In at least one implementation, the threshold number of short read alignments is a first pre-determined value that is calibrated to distinguish true structural variants with an adequate or desired precision, e.g., based on reference data for which the structural variants are known. As such, the short read sequencing data 130 may be used for orthogonal validation of structural variants identified based on the long read sequencing data 134.

As used herein, a “graph-based reference” may refer to a data structure that represents genomic sequences and their variations as a graph. In this representation, nodes typically correspond to sequences of nucleotides, while edges represent connections between these sequences. This structure allows for the incorporation of known genetic variations, such as single nucleotide polymorphisms, insertions, deletions, and structural variants, into the reference itself. Graph-based references may provide a more comprehensive representation of genomic diversity compared to traditional linear references, which may improve the accuracy of read mapping and variant calling.

For example, the integrated variant calling module 156 may use the long read sequencing data 134 for initial structural variant detection. The alignment module 138 may then create a personalized graph-based reference (e.g., an augmented reference) that incorporates these detected variants, as mentioned above. The short read sequencing data 130 may be mapped to this graph-based reference. Subsequently, the integrated variant calling module 156 may refine structural variant calls and genotypes based on evidence from the short read sequencing data 130. This integrated approach may improve both the sensitivity and specificity of the variant call 164 compared to using either the short read sequencing data 130 or the long read sequencing data 134 alone.

Additionally, or alternatively, short variants determined by the one or more short variant calling algorithms 158 from the short read sequencing data 130 may be filtered (e.g., to increase precision) by using a set of features that come from both the long and the short read sequencing data 134. Similar to that described above, the short variants and the reference sequence 142 may be used to generate the variation graph, and the long read sequencing data 134 may be aligned to the variation graph (e.g., rather than the reference sequence 142). Short variants at locations on the variation graph that are supported by at least a threshold number of long read alignments are considered to be validated. In at least one implementation, the threshold number of long read alignments is a second pre-determined value that is calibrated to distinguish true short variants with an adequate or desired precision, e.g., based on reference data for which the short variants are known. The second pre-determined value may be the same as or different than the first pre-determined value of the threshold number of short read alignments. For example, the threshold number of long read alignments may be greater than the threshold number of short read alignments. Alternatively, the threshold number of long read alignments may be less than the threshold number of short read alignments or equal to the threshold number of short read alignments. As such, the long read sequencing data 134 may be used for orthogonal validation of short variants identified based on the short read sequencing data 130. Additionally, or alternatively, the long read sequencing data 134 may be used to connect nearby variants, which may improve phasing of short variants, of structural variants, or of both types.

In at least one variation, the one or more refinement algorithms 162 include functionality for correcting errors in the long read sequencing data 134 based on the short read sequencing data 130. By way of example, some long read sequencing techniques have a higher error rate than short read sequencing techniques. As such, the one or more refinement algorithms 162 may align the short read sequencing data 130 to the long read sequencing data 134 to correct errors in the long read sequencing data 134. The one or more structural variant calling algorithms 160 may then perform structural variant calling using the error corrected long read sequencing data 134. Correcting errors in the long read sequencing data 134 based on the short read sequencing data 130 may enable the long read coverage to be further reduced while maintaining a desired or adequate structural variant calling precision and/or sensitivity.

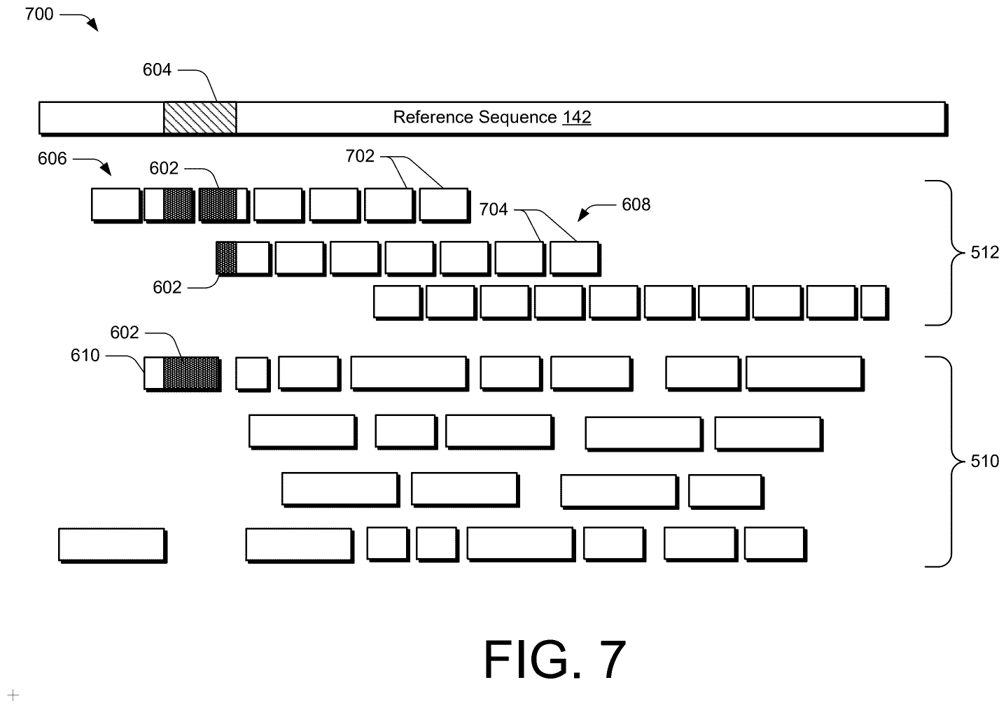

In at least one other variation, the one or more refinement algorithms 162 include functionality to bioinformatically divide the long read sequencing data 134 into short read lengths for evaluation using the one or more short variant calling algorithms 158. Doing so may maintain the alignment of the long read sequencing data 134 while making the long read sequencing data 134 usable for short read variant calling. By way of example, the one or more refinement algorithms 162 may be trained to identify locations at which to split the long read sequencing data 134 into synthetic (or “virtual”) short reads. For instance, the long read sequencing data 134 may be divided into synthetic short reads of varying length based on identification of an appropriate division location within a short read length range (e.g., a range from tens to hundreds of nucleotides), such as will be further described with respect to FIG. 7. In one or more implementations, the one or more short variant calling algorithms 158 may be further tuned or adapted (e.g., by the one or more refinement algorithms 162) to long read error types in order to account for differences between the long read (e.g., synthetic short read) and short read data. Other variations are possible without departing from the scope of the described techniques.

In one or more implementations, the integrated variant calling module 156 may employ genotype-aware structural variant calling techniques. This approach may enhance the accuracy and completeness of variant detection by considering both the presence of a variant (allele) and its copy number (genotype) in the genome. As used herein, the term “genotype-aware” refers to a method of variant calling that considers not only the presence or absence of a structural variant, but also the number of copies of that variant present in the genome. In diploid organisms like humans, this typically means determining whether an individual has 0, 1, or 2 copies of a particular variant.

The genotype-aware approach leverages the complementary strengths of the short read sequencing data 130 and the long read sequencing data 134. The long read sequencing data 134 may provide increased detection of structural variants, particularly in complex genomic regions, while the short read sequencing data 130 may offer high accuracy for genotyping due to more uniform coverage and lower error rates.

To evaluate the performance of genotype-aware structural variant calling, the integrated variant calling module 156 may employ specialized statistical measures. The genotype-aware versions of precision, recall, and F1 scores are computed using the same formulas as the typical allele-level matching versions for structural variants. However, when computing the number of true positives, these statistics also rely on the correct genotype for a variant call to be considered correct overall, in addition to the correct allele. These genotype-aware statistics provide a more stringent and comprehensive assessment of variant calling performance, as they account for both the accurate detection of the variant and the correct determination of its copy number in the genome. By incorporating genotype-aware structural variant calling into the integrated short read and long read sequencing approach described herein, the integrated variant calling module 156 may achieve improved accuracy in variant detection and characterization, which may result in more reliable genomic analyses and interpretations.

The client device 104 is shown displaying, via a display device 166, the sequence 140, or a portion thereof, as well as the variant call 164. By way of example, the display device 166 may display a portion of the sequence 140 as a string of characters representing the sequence of nucleotides in the portion. Additionally, or alternatively, the display device 166 may display the sequence 140 as a visual representation of the short reads and the long reads aligned with the reference sequence 142 along with an indication of a nucleotide identified at a specific location. The variant call 164 may be displayed by the display device 166 as a visual representation of genomic location(s) where variant(s) are present and/or as a list of detected variant(s) and their genomic location(s). It is to be appreciated that the variant call 164 and the sequence 140 are also stored in memory, in a single data file or multiple data files, for subsequent access.

In this way, the sequencing integrator 106 enables structural variants to be accurately identified without losing information regarding short variants and with reduced costs and/or use of sequencing resources compared with traditional long read sequencing. Moreover, the sequencing integrator 106 enables short variants to be accurately and efficiently identified in genomic regions that are difficult to map using traditional short read sequencing. As still another example, the sequencing integrator 106 enables the generation of high coverage, fully assembled mitochondrial haplotypes with reduced cost (e.g., compared to higher coverage short read and/or long read sequencing techniques) by combining the short read sequencing data 130 and the long read sequencing data 134. Overall, the sequencing integrator 106 can provide customizable sequencing coverage based on desired sequencing parameters, a sequencing target, and/or a cost target. For example, the sequencing integrator 106 supports the use of lower coverage short read sequencing in combination with low coverage long read sequencing without sacrificing sequencing sensitivity and precision. As a result, sequencing resources are more efficiently utilized without sacrificing an amount and quality of genomic information obtained.

### Integrated Short and Long Read Sequencing

FIG. 2 depicts an example 200 of an implementation of the protocol generation module 118 of FIG. 1 in greater detail. In particular, the illustrated example 200 represents a framework according to which the protocol generation module 118 generates the short read protocol 124 and the long read protocol 126 based on input regarding the sequencing event 114.

The illustrated example 200 includes, from FIG. 1, the protocol generation module 118, including the coverage modulation engine 120 and the sequencing protocol generator 122. The illustrated example 200 further includes, from FIG. 1, the short read protocol 124 and the long read protocol 126. Additional details regarding the coverage modulation engine 120 are discussed with respect to FIG. 3.

In the illustrated example 200, the coverage modulation engine 120 of the protocol generation module 118 receives an input 202. In at least one implementation, the input 202 is received from a user, e.g., as submitted via the client device 104 running the application 112, such as shown in FIG. 1. The input 202 comprises one or more inputs, such as a sequencing target 204, a performance constraint 206, and/or a resource constraint 208. By way of example, the sequencing target 204 is a type of variant that is to be detected (e.g., a variant target), such as an SNP, an INDEL, a CNV, an inversion, and so forth. Additionally, or alternatively, the sequencing target 204 includes a specific region of the genome (e.g., a gene region) to be investigated and/or a sequencing technology to be used. The sequencing technology, for instance, defines a selection of the short read technology and/or long read technology to be used. Additionally, or alternatively, the sequencing target 204 includes a disease target or a class of diseases that are being targeted, which may provide information regarding the gene region(s) and/or the variant target(s) of the sequencing. It is to be appreciated that in one or more implementations, the sequencing target 204 includes a selection of more than one target, such as one or more variant targets, one or more gene regions, and/or one or more disease targets, alone or in various combinations.

The performance constraint 206 indicates one or more desired (e.g., targeted) performance metrics of the sequencing event 114, such as with respect to the variant call 164. In the illustrated example 200, the one or more performance metrics are selected from a group comprising an accuracy metric 210, a sensitivity metric 212, and a precision metric 214. The accuracy metric 210 refers to how well the determined nucleotide bases in sequenced data match a true or expected sequence. For instance, the accuracy metric 210 is determined for samples for which the sequence is known. The sensitivity metric 212 refers to the ability to correctly identify true positive variants in a sequenced sample. The precision metric 214 refers to a proportion of true positive calls among all positive calls made for a sequenced sample. It is to be appreciated that the performance constraint 206 may be related to sequencing data obtained for reference samples having known sequences and known variants using a plurality of different sequencing coverage combinations, such as will be elaborated with respect to FIG. 3, and these metric values may be used by the coverage modulation engine 120 to predict sequencing performance for an unknown sample. Moreover, rather than providing a single value, in at least one implementation, the accuracy metric 210, the sensitivity metric 212, and/or the precision metric 214 is given as a range of values.

Additionally, or alternatively, the performance constraint 206 corresponds to a specific type of variant and/or a certain genomic region (e.g., a targeted chromosome or location on a chromosome) of the variant. By way of example, the performance constraint 206 may include a first targeted sensitivity for identifying a SNP in a first genomic region and a second targeted sensitivity for identifying an INDEL in a second genomic region. As such, it is to be appreciated that the accuracy metric 210, the sensitivity metric 212, and/or the precision metric 214 may include more than one input, and different inputs may be associated with different variant and/or genomic locations.

As may be appreciated from the above discussion, in various scenarios, the performance constraint 206 includes only the accuracy metric 210, only the sensitivity metric 212, only the precision metric 214, the accuracy metric 210 in combination with the sensitivity metric 212, the accuracy metric 210 in combination with the precision metric 214, the sensitivity metric 212 in combination with the precision metric 214, or the accuracy metric 210 in combination with both of the sensitivity metric 212 and the precision metric 214. As such, the performance constraint 206 includes one or more or each of the accuracy metric 210, the sensitivity metric 212, and the precision metric 214 in various combinations.

The resource constraint 208 is one or both of a monetary cost (e.g., monetary resource) constraint and a sequencing resource cost constraint. By way of example, the monetary cost is a price per base or an overall cost of sequencing a sample. The sequencing resource cost is an amount of data generated by sequencing the sample in the sequencing event 114 and/or a run time of the sequencing event 114, for example. Alternatively, or in addition, the sequencing resource is a throughput capacity of a sequencer to be used for the sequencing event 114.

In at least one variation, the input 202 is at least partially generated according to pre-programmed instructions. As a non-limiting example, the sequencing target 204 is input by the user or selected from a plurality of sequencing target options, and the performance constraint 206 is automatically populated with a pre-programmed suggestion based on the sequencing target 204. Alternatively, or in addition, the resource constraint 208 is input by the user, and the performance constraint 206 is automatically populated with a pre-programmed suggestion based on the given resource constraint 208. In at least one implementation, the performance constraint 206, even when automatically populated, is further adjustable based on additional user input.

Moreover, in one or more implementations, a warning is output by the protocol generation module 118 in response to incompatible inputs being received or otherwise selected. By way of example, the warning may indicate that a desired performance constraint 206 is outside of an available range for the given resource constraint 208. As another non-limiting example, the warning may indicate that the resource constraint 208 is outside of an available range for the given sequencing target 204. As such, in at least one implementation, the protocol generation module 118 includes user interface functionality for guiding the user to provide input 202 that will enable generation of a valid sequencing protocol for the given inputs.

In accordance with the described techniques, the coverage modulation engine 120 receives the input 202 and selects a short read sequencing coverage 216 (abbreviated as “SR sequencing coverage” in the figures) and a long read sequencing coverage 218 (abbreviated as “LR sequencing coverage” in the figures) in response thereto. By way of example, the short read sequencing coverage 216 and the long read sequencing coverage 218 are levels of coverage that are selected (e.g., by the coverage modulation engine 120) with respect to each other and based on the input 202. As will be elaborated below with respect to FIG. 3, in at least one implementation, the coverage modulation engine 120 generates and utilizes pre-defined relationships between the sequencing coverage and a range of values for the various parameters of the input 202 for different combinations of short read sequencing coverage and long read sequencing coverage. The coverage modulation engine 120 references these pre-defined relationships to determine the short read sequencing coverage 216 and the long read sequencing coverage 218 that will provide sequencing results consistent with the input 202. As such, the input 202 functions as a modulator for modulating levels of coverage and targeting for each sequencing method and/or as a switch to select specific short/long read technologies.

In one or more implementations, the coverage modulation engine 120 includes a multi-objective algorithm that is configured to consider multiple different inputs (e.g., of the input 202) at the same time to determine the short read sequencing coverage 216 and the long read sequencing coverage 218. In some scenarios, the multiple different inputs may present objectives that are competing and/or in conflict with each other, and the multi-objective algorithm may be configured to output a set of solutions (e.g., the short read sequencing coverage 216 and the long read sequencing coverage 218) that represent a trade-off or compromise among the competing/conflicting objectives. In at least one implementation, the multi-objective algorithm includes a ranked hierarchy of the inputs, which may be used to prioritize one type of input over another. By way of example, the ranked hierarchy may rank the sequencing target 204, the performance constraint 206, and the resource constraint 208 relative to each other. In at least one variation, however, the multi-objective algorithm does not use a ranked hierarchy of the inputs.

In one or more implementations, the long read sequencing coverage 218 includes more than one long read sequencing coverage value, such as when multiple different long read sequencing techniques are to be used. For example, the long read sequencing coverage 218 may include a first long read sequencing coverage value for a first long read sequencing technique and a second long read sequencing value for a second long read sequencing technique. In such scenarios, the coverage modulation engine 120 may determine the first long read sequencing coverage and the second long read sequencing coverage by identifying a mixture of the different long read sequencing techniques, in combination with the short read sequencing coverage 216, that will provide sequencing results consistent with the input 202.

In general, traditional short read sequencing is performed at 30× to 40× coverage or more for short variant detection, and traditional long read sequencing is performed at 30× to 40× coverage or more for structural variant detection. Because the techniques described herein advantageously augment short read sequencing and long read sequencing with respect to each other, the short read sequencing coverage 216 may be less than 30×, such as 25× or even less than 25× (e.g., 22× or 15×). Similarly, the long read sequencing coverage 218 may be less than 30×, such as 10× or even less than 10× (e.g., 4×).

In order to increase an amount of information provided per dollar spent, and because long read sequencing is more expensive than short read sequencing, the long read sequencing coverage 218 may be less than the short read sequencing coverage 216, at least in one or more implementations. By way of example, the long read sequencing coverage 218 is reduced (e.g., minimized) based on the sequencing target 204 to a value that is expected, based on long read sequencing data of reference samples, to cover hard-to-map regions and structural variants that are not covered by short reads, and the short read sequencing coverage 216 is modulated to a level that is that within the performance constraint 206 and the resource constraint 208 in combination with the reduced (e.g., minimized) long read sequencing coverage 218.

In the illustrated example 200, the sequencing protocol generator 122 receives the short read sequencing coverage 216 and the long read sequencing coverage 218 and generates the short read protocol 124 and the long read protocol 126. This includes, for example, adjusting the short read protocol 124 based on the short read sequencing coverage 216, such as to reduce or establish a number of reads performed by the short read sequencer 128, an amount of data generated by the short read sequencer 128, and/or a sequencing time of the short read sequencer 128 with respect to a nucleic acid sample. Similarly, generating the long read protocol 126 may include reducing or establishing a number of reads performed by the long read sequencer 132, an amount of data generated by the long read sequencer 132, and/or a sequencing time of the long read sequencer 132 with respect to the nucleic acid sample.

In this way, the protocol generation module 118 identifies sequencing coverage levels for short read sequencing and long read sequencing that will result in variant calls for the sequencing target 204 having a desired level of sensitivity and/or precision that is at least adequate for the intended detection. By way of example, a sensitivity of the variant call is in a first range between 0.9900 and 0.9999, or between 0.9925 and 0.9990, or between 0.9950 and 0.9977 for an SNP, while a precision of the variant call is in a second range between 0.9700 and 0.9999, or between 0.9800 and 0.9970, or between 0.9850 and 0.9960 for the SNP. As another example, the sensitivity of the variant call is in a third range between 0.9700 and 0.9999, or between 0.9800 and 0.9970, or between 0.9850 and 0.9960 for an INDEL, while the precision of the variant call is in a fourth range between 0.9700 and 0.9990, or between 0.9800 and 0.9985, or between 0.9970 and 0.9981 for the INDEL. As still another example, the sensitivity of the variant call is in a fifth range between 0.20 and 0.80, or between 0.22 and 0.80, or between 0.50 and 0.70 for a structural variant, while the precision of the variant call is in a sixth range between 0.50 and 0.90, or between 0.60 and 0.85, or between 0.65 and 0.80 for the structural variant. It is to be appreciated that the above described sensitivity and precision values may be achieved according to the techniques described herein while the short read sequencing coverage is less than thirty (e.g., a short read coverage value between five and thirty, or between ten and twenty-five, or between fifteen and twenty-two) and the long read sequencing coverage is less than thirty (e.g., in a long read coverage value between two and thirty, or between two and eight, or between three and five).

FIG. 3 depicts an example 300 of an implementation of the coverage modulation engine 120 of FIG. 1 in greater detail. In particular, the illustrated example 300 represents a framework according to which the coverage modulation engine 120 selects the short read sequencing coverage 216 and the long read sequencing coverage 218 introduced with respect to FIG. 2.

In the illustrated example, the coverage modulation engine 120 includes a coverage relationship generator 302, a coverage relationship library 304, and a coverage selector 306. The coverage relationship generator 302 represents functionality of the coverage modulation engine 120 to determine or otherwise generate relationships between coverage depths and various customizable sequencing parameters, such as those provided with respect to the input 202 and described above with reference to FIG. 2, that are used to modulate sequencing coverage. The coverage relationship library 304 represents the relationships generated by the coverage relationship generator 302. By way of example, the coverage relationship library 304 includes one or more data suppliers, such as look-up tables, graphs, formulas, and/or models, that are generated or otherwise determined by the coverage relationship generator 302 and that relate the customizable sequencing parameters to coverage depth for a plurality of combinations of short read and long read sequencing coverages. The coverage selector 306 represents functionality of the coverage modulation engine 120 to select and output the short read sequencing coverage 216 and the long read sequencing coverage 218 based on the input 202 by referencing the coverage relationship library 304.

In the example implementation illustrated in FIG. 3, the coverage relationship generator 302 generates the coverage relationship library 304 based on reference sequencing data 308. The reference sequencing data 308 comprises sequencing data generated through long read and short read sequencing data acquired for a reference sample (or samples) having a known sequence at a plurality of different coverages and/or using a plurality of different sequencing techniques. For example, the reference sequencing data 308 includes sequencing data generated via a plurality of different sequencing events (e.g., “runs” or “experiments”), represented in FIG. 3 as a first reference sequencing event 310 (e.g., “reference sequencing event 1”), a second reference sequencing event 312 (e.g., “reference sequencing event 2”), and an nth reference sequencing event 314 (e.g., “reference sequencing event N”). By way of example, the first reference sequencing event 310 differs from others of the reference sequencing data 308 with respect to one or more or each of a sequencing technique used to generate the data, a coverage level of the sequencing, a sequencer and/or sequencing center used to generate the data, a type of variant targeted by the sequencing event, and an identity of the reference sample. The reference sequencing data 308 includes both short read sequencing data and long read sequencing data. By way of example, the first reference sequencing event 310 comprises short read sequencing data for a particular reference, and the second reference sequencing event 312 comprises long read sequencing data for the particular reference.

Because the sequence of the reference is known exactly or known within some degree of error, sequences generated from the reference sequencing data 308 as well as a sensitivity and precision of variant calls can be determined and used by the coverage relationship generator 302 to generate the coverage relationship library 304. By way of example, the coverage relationship generator 302 is configured to generate relationships between short read and long read coverage with respect to base calling accuracy, variant call precision, variant call sensitivity (e.g., recall), and sequencing cost. These relationships are represented in FIG. 3 as a first precision relationship 316 (e.g., “precision relationship 1”), a second precision relationship 318 (e.g., “precision relationship 2”), an nth precision relationship 320 (e.g., “precision relationship N”), a first sensitivity relationship 322 (e.g., “sensitivity relationship 1”), a second sensitivity relationship 324 (e.g., “sensitivity relationship 2”), an nth sensitivity relationship 326 (e.g., “sensitivity relationship N”), a first accuracy relationship 328 (e.g., “accuracy relationship 1”), a second accuracy relationship 330 (e.g., “accuracy relationship 2”), an nth accuracy relationship 332 (e.g., “accuracy relationship N), a first cost relationship 334 (e.g., “cost relationship 1”), a second cost relationship 336 (e.g., “cost relationship 2”), and an nth cost relationship 338 (e.g., “cost relationship N”).

By way of example, the first precision relationship 316 defines the variant call precision over a range of short read sequencing coverage when combined with a first long read sequencing coverage, whereas the second precision relationship 318 defines the variant call precision over the range for short read sequencing coverage when combined with a second long read sequencing coverage. As such, the different precision relationships vary from each other with respect to one or more or each of long read sequencing coverage, short and/or long read sequencing technique, sequencer(s), sequencing target, and reference used.

Similarly, the first sensitivity relationship 322, the second sensitivity relationship 324, the nth sensitivity relationship 326, and any other sensitivity relationships included in the coverage relationship library 304 define the variant call sensitivity over the range of short read sequencing coverage and differ from each other with respect to one or more or each of the long read sequencing coverage, the short and/or long read sequencing technique, the sequencer(s), the sequencing target, and the reference used. The first accuracy relationship 328, the second accuracy relationship 330, the nth accuracy relationship 332, and any other accuracy relationships included in the coverage relationship library 304 define the base calling accuracy over the range of short read sequencing coverage and differ from each other with respect to one or more or each of the long read sequencing coverage, the short and/or long read sequencing technique, the sequencer(s), the sequencing target, and the reference used.

The first cost relationship 334, the second cost relationship 336, and the nth cost relationship 338 define a resource cost with respect to short read or long read sequencing coverage. By way of example, the first cost relationship 334 defines a cost per base over the range of short range sequencing coverage using a first short read sequencing technique, the second cost relationship 336 defines a cost per base over the range of short read sequencing coverage using a second short read sequencing technique, the nth cost relationship 338 defines a cost per base over a range of long read sequencing coverage using a first long read sequencing technique, and so forth.

It is to be appreciated that the above examples are illustrative, and more, fewer, or different coverage relationships may be included in the coverage relationship library 304 without departing from the scope of the described techniques. Moreover, although the coverage relationship library 304 is shown as integrated with the coverage modulation engine 120, in at least one variation, the coverage relationship library 304 is stored separately from the coverage modulation engine 120 and accessed by the coverage modulation engine 120, such as by the coverage relationship generator 302, in order to customize short read and long read sequencing coverage.

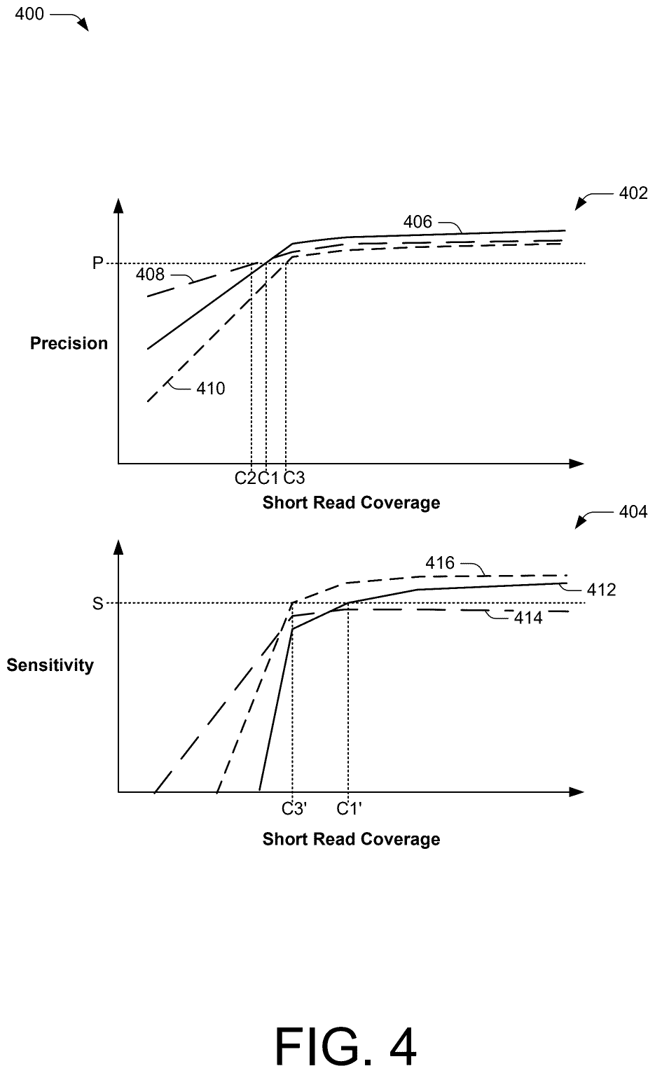

FIG. 4 depicts a simplified illustrative example 400 of selecting short read and long read sequencing coverage based on modulation inputs. The example 400 depicts a first graph 402 of a plurality of relationships between short read coverage and precision (e.g., variant call precision) and a second graph 404 of a plurality of relationships between the short read coverage and sensitivity (e.g., of variant recall). By way of example, the first graph 402 and the second graph 404 are generated by the coverage relationship generator 302 and stored in the coverage relationship library 304 for reference by the coverage selector 306 in determining the short read sequencing coverage 216 and the long read sequencing coverage 218 based on the input 202, such as depicted with respect to FIG. 3 and described above.

In the example 400, the first graph 402 includes a first precision plot 406 that defines a relationship between the short read coverage and the precision at a first long read coverage, a second precision plot 408 that defines a relationship between the short read coverage and the precision at a second long read coverage, and a third precision plot 410 that defines a relationship between the short read coverage and the precision at a third long read coverage. The coverage modulation engine 120 receives, as the input 202, a precision value P. The precision value P corresponds to a short read coverage value C1 at the first long read coverage, a short read coverage value C2 at the second long read coverage, and a short read coverage value C3 at the third long read coverage. That is, the short read coverage value C1 corresponds to a value of the first precision plot 406 at the precision value P, the short read coverage value C2 corresponds to a value of the second precision plot 408 at the precision value P, and the short read coverage value C3 corresponds to a value of the third precision plot 410 at the precision value P. In the depicted example 400, the short read coverage value C2 is the smallest, and the short read coverage value C3 is the largest.

The second graph 404 includes a first sensitivity plot 412 that defines a relationship between the short read coverage and the sensitivity at the first long read coverage, a second sensitivity plot 414 that defines a relationship between the short read coverage and the sensitivity at the second long read coverage, and a third sensitivity plot 416 that defines a relationship between the short read coverage and the sensitivity at the third long read coverage. As such, the first sensitivity plot 412 is derived from the same sequencing data as the first precision plot 406, the second sensitivity plot 414 is derived from the same sequencing data as the second precision plot 408, and the third sensitivity plot 416 is derived from the same sequencing data as the third precision plot 410 in the present example implementation.

The coverage modulation engine 120 also receives, as the input 202, a sensitivity value S. The sensitivity value S corresponds to a short read coverage value C1′ at the first long read coverage and a short read coverage value C3′ at the third long read coverage. That is, the short read coverage value C1′ corresponds to a value of the first sensitivity plot 412 at the sensitivity value S, and the short read coverage value C3′ corresponds to a value of the third sensitivity plot 416 at the sensitivity value S. The second sensitivity plot 414 does not intersect with the sensitivity value S. As such, the second long read coverage is unable to provide the requested sensitivity value S with any combination of short read coverage. The second long read coverage is thus excluded from being selected as the long read sequencing coverage 218.

In the present example 400, the precision value P and the sensitivity value S represent a lower limit of an acceptable precision or sensitivity value range, respectively. For example, it is desired to achieve a variant call precision of no less than the precision value P and a variant call sensitivity of no less than the sensitivity value S. In order to provide both the requested precision value P and the requested sensitivity value S, in at least one implementation, the coverage selector 306 compares the short read coverage value C1 with the short read coverage value C1′ and compares the short read coverage value C3 with the short read coverage value C3′. The short read coverage value C1 is less than the short read coverage value C1′ and would not provide the requested sensitivity value S. In contrast, the short read coverage value C1′ would provide the requested precision value P. Accordingly, the short read coverage value C1′ is selected to be combined with the first long read coverage as a first short read and long read coverage combination. Similarly, the short read coverage value C3 is less than the short read coverage value C3′ and would not provide the requested sensitivity value S, but the short read coverage value C3′ would provide the requested precision value P. Thus, the short read coverage value C3′ is selected to be combined with the third long read coverage as a second short read and long read coverage combination.

In selecting between the first short read and long read coverage combination and the second short read and long read coverage combination, the coverage selector 306 may take into account resource cost (e.g., monetary cost and/or resource usage) and select the combination having the lowest resource cost for the short read sequencing coverage 216 and the long read sequencing coverage 218. As such, the coverage selector 306 may compare a combined cost of the first short read and long read coverage combination and the second short read and long read coverage combination in selecting the short read and long read coverage combination.

As an illustrative example where the first long read coverage is 4×, the short read coverage value C1′ is 22×, the third long read coverage is 8×, and the short read coverage value C3′ is 15×, the coverage selector 306 may select the first short read and long read coverage combination due to the long read sequencing being more expensive than short read sequencing. In this example scenario, a cost difference between providing 8× long read coverage and 4× long read coverage is greater than a cost difference between providing 22×short read coverage and 15× short read coverage. As such, the first short read and long read coverage combination is selected because it is demonstrated to provide comparable or adequate precision and sensitivity for a given sequencing target compared with the second short read and long read coverage combination in a more cost-effective and resource-efficient manner.

It is to be appreciated that still other inputs may be used in the coverage selection process, such as those described above with respect to FIG. 2. As such, the example 400 provides a simplified illustrated example of processes that may be performed by the coverage selector 306 in selecting the short read sequencing coverage 216 and the long read sequencing coverage 218, and additional or alternative inputs and processes may be used without departing from the scope of the described techniques.

FIG. 5 depicts a workflow 500 in an example implementation of variant calling by the sequencing integrator 106 of FIG. 1. The illustrated example 200 includes, from FIG. 1, the protocol generation module 118, including the coverage modulation engine 120 and the sequencing protocol generator 122. For instance, the workflow 500 outlines a sequencing pipeline for the sequencing event 114. The workflow 500 includes, from FIG. 1, the alignment module 138 and the integrated variant calling module 156. Although not specifically depicted for illustrative simplicity, it is to be appreciated that the workflow 500 also includes the protocol generation module 118, as indicated by the inclusion of the short read protocol 124 and the long read protocol 126.

A biological sample 502 is processed to prepare a nucleic acid sample, shown in FIG. 5 as DNA 504. By way of example, the DNA 504 (or another nucleic acid) is isolated from the biological sample 502 using a DNA extraction technique. The biological sample 502 comprises, for example, blood, tissue, saliva, or another source of cells from an organism (e.g., individual) of interest. Short read sample preparation 506 (e.g., “SR preparation”) is performed on at least a first portion of the DNA 504, and long read sample preparation 508 (e.g., “LR preparation”) is performed on at least second portion of the DNA 504. In at least one implementation, the short read sample preparation 506 includes fragmenting the DNA 504 into short DNA fragments (e.g., typically between approximately 50 base pairs and several hundred base pairs) and attaching sequencing adapters to the short DNA fragments that are used by the short read sequencer 128 to facilitate sequencing. The short read sample preparation 506 may be adapted to the short read protocol 124. Similarly, in at least one implementation, the long read sample preparation 508 includes fragmenting the DNA 504 into long DNA fragments (e.g., typically thousand to hundreds of thousands of base pairs) and attaching different sequencing adapters to the long DNA fragments that are used by the long read sequencer 132 to facilitate sequencing. The long read sample preparation 508 may be adapted to the long read protocol 126.

In at least one implementation, the short read sample preparation 506 is performed independently from the long read sample preparation 508 such that there is no direct correspondence between the short DNA fragments and the long DNA fragments. In variations, however, the short read sample preparation 506 utilizes the long DNA fragments prepared in the long read sample preparation 508. By way of example, the long DNA fragments may be amplified and tagged, e.g., with a tag “barcode” that is different for different long DNA fragments, and a portion of the barcoded long DNA fragments may be further fragmented into the short DNA fragments via the short read sample preparation 506. Doing so may enable correspondence to be maintained between the long DNA fragments and the short DNA fragments across sequencing techniques for subsequent alignment that has reduced ambiguity. For instance, a short read associated with a first barcode may be efficiently aligned with a long read associated with the first barcode.

Short read sequencing is performed by the short read sequencer 128 on the short DNA fragments prepared via the short read sample preparation 506 according to the short read protocol 124, which is generated based on the short read sequencing coverage 216 determined by the coverage modulation engine 120. In at least one implementation, the short read sequencer 128 employs fluorescence-based detection to determine an order of nucleotides in the short DNA fragments. By way of example, labeled nucleotides (e.g., fluorescently labeled or otherwise labeled nucleotides) are added to the short DNA fragments, and as each nucleotide is incorporated, a signal (e.g., a fluorescent signal) is emitted, allowing the determination of the sequence for a plurality of short DNA fragments in parallel. The short read sequencer 128 generates and outputs the short read sequencing data 130 based on the fluorescent signals.

Long read sequencing is performed by the long read sequencer 132 on the long DNA fragments prepared via the long read sample preparation 508 according to the long read protocol 126, which is generated based on the long read sequencing coverage 218 determined by the coverage modulation engine 120. In at least one implementation the long read sequencer 132 directly reads the sequence of a given large DNA fragment as it passes through a pore (e.g., nanopore) or is captured by a zero-mode waveguide. The long read sequencer 132 generates and outputs the long read sequencing data 134 based on the directly read sequences.

As discussed above with respect to FIG. 1, the alignment module 138 receives the short read sequencing data 130 and the long read sequencing data 134 to determine the sequence 140 based on the reference sequence 142. In at least one implementation, the alignment module 138 generates aligned short reads 510 (e.g., “aligned SRs”) using the one or more short read alignment algorithms 148 and generates aligned long reads 512 using the one or more long read alignment algorithms 152. Moreover, in accordance with the techniques described herein, the aligned short reads 510 may be adjusted based on the aligned long reads 512, or vice versa, via the composite alignment module 154. For instance, the composite alignment module 154 generates an augmented reference 514 based on the aligned long reads 512 and uses the augmented reference 514 to adjust (e.g., correct) the aligned short reads 510. Alternatively, or in addition, the short read sequencing data 130 is directly aligned to the augmented reference 514 rather than aligned to the reference sequence 142 in generating the aligned short reads 510. In scenarios where barcoding is used, the short read sequencing data 130 may be aligned to the aligned long reads 512 in the augmented reference 514 based on a short read having a same barcode as a long read in addition to an order of nucleotides in the short read compared relative to the long read.

In at least one implementation, the alignment module 138 outputs aligned reads 516, which comprise the aligned short reads 510 and the aligned long reads 512 relative to the reference sequence 142, and provides the sequence 140. For instance, the sequence 140 is reconstructed by assembling the aligned short reads 510 and aligned long reads 512 into a longer, contiguous sequence based on overlapping portions of the aligned short reads 510 and/or the aligned long reads 512.

The aligned reads 516 (or the sequence 140 itself) are compared to the reference sequence 142 by the integrated variant calling module 156 to output the variant call 164. In at least one implementation, the one or more short variant calling algorithms 158 output preliminary short variants 518, and the one or more structural variant calling algorithms 160 output preliminary structural variants 520 via a separate process. In such implementations, the one or more refinement algorithms 162 correct and/or validate (e.g., verify) the preliminary short variants 518 based on the preliminary structural variants 520. Additionally, or alternatively, the one or more refinement algorithms 162 correct and/or validate the preliminary structural variants 520 based on the preliminary short variants 518. The one or more refinement algorithms 162 output the variant call 164, which includes corrected and/or validated short variants of the preliminary short variants 518, when identified, as well as corrected and/or validated structural variants of the preliminary structural variants 520, when identified.

It is to be appreciated that as used herein, the term “align” and its conjugates is not limited to an exact 1:1 alignment between sequences. Rather, alignment is accomplished with a degree of accuracy that is adequate or desired based on its intended purpose (e.g., sufficient accuracy to identify variants with a targeted sensitivity and precision).

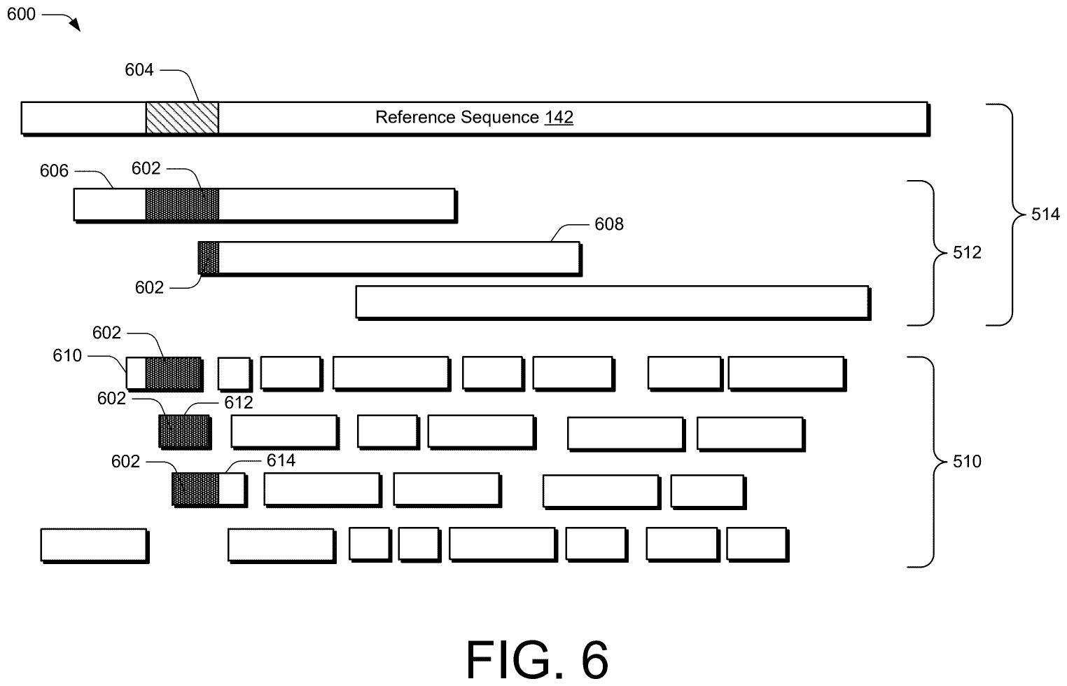

FIG. 6 shows an example of an alignment 600 in a first example of integrating short read and long read sequencing for genomic variant detection. The alignment 600 is a simplified example that depicts the aligned short reads 510 and the aligned long reads 512 with respect to the reference sequence 142. Together, the aligned long reads 512 and the reference sequence 142 form the augmented reference 514, and individual short reads are aligned to the augmented reference 514.

In the depicted example, the sequenced sample (e.g., the biological sample 502 of FIG. 5) includes a variant sequence 602 corresponding to a structural variant at a genomic location 604 on the reference sequence 142. By way of example, the variant sequence 602 is an inversion of the reference sequence 142 at the genomic location 604. A first long read 606 includes an entirety of the variant sequence 602, and a second long read 608 includes a portion of the variant sequence 602. The first long read 606 and the second long read 608 also include one or more long sequence fragments that are not included in the variant sequence 602 and thus map to the reference sequence 142. As such, the first long read 606 and the second long read 608 may be aligned to the reference sequence 142 (e.g., via the one or more long read alignment algorithms 152 of the long read alignment module 150) with high confidence despite differing at the genomic location 604.

A first short read 610, a second short read 612, and a third short read 614 also include varying portions of the variant sequence 602. In contrast to the first long read 606 and the second long read 608, the first short read 610, the second short read 612 and the third short read 614 cannot be mapped to the reference sequence 142 with high confidence. For instance, the second short read 612 includes a portion of the variant sequence 602 without any other sequence portion that relates its position to the reference sequence 142, and the first short read 610 and the third short read 614 include relatively small sequence portions that match the reference sequence 142. As such, the one or more short read alignment algorithms 148 of the short read alignment module 146 may be unable to map the first short read 610, the second short read 612, and the third short read 614 directly to the reference sequence 142.

Instead, in at least one implementation, the composite alignment module 154 maps the first short read 610, the second short read 612, and the third short read 614 to the augmented reference 514, e.g., to the aligned first long read 606 and the aligned second long read 608 based on the variant sequence 602. As such, the structural variant associated with the variant sequence 602 may be identified (e.g., by the integrated variant calling module 156) with reduced long read coverage compared to conventional structural variant detection techniques.

In an alternative example scenario, the genomic location 604 is an intrinsic hard-to-map region of the reference sequence 142. For example, the genomic location 604 may be a highly repetitive region where short reads (e.g., the second short read 612) cannot be uniquely aligned and/or cannot be aligned with acceptable confidence (e.g., as dictated by parameters of the one or more short read alignment algorithms 148). In such an example scenario, the aligned long reads 512 increase a coverage of the genomic location 604, thus enabling variants in the genomic location 604 to be identified with higher sensitivity and precision than when the short read sequencing data 130 or the long read sequencing data 134 are used alone. By way of example, SNP and INDEL recall may be improved for the genomic location 604 compared to using traditional short read sequencing alone.

It is to be appreciated that the above alignment 600 is simplified, and a size of the aligned short reads 510 relative to the aligned long reads 512 and relative to the reference sequence 142, as well as their quantities, is not to scale.

FIG. 7 shows an example of an alignment 700 in a second example of integrating short read and long read sequencing for genomic variant detection. The alignment 700 is a simplified example that depicts the aligned short reads 510 and the aligned long reads 512 with respect to the reference sequence 142. The alignment 700 is similar to the alignment 600 of FIG. 6, and so the following description highlights the differences between the alignment 700 and the alignment 600.

In the alignment 700, once the long read alignment is performed (e.g., by the one or more long read alignment algorithms 152) to produce the aligned long reads 512, the aligned long reads 512 are fragmented into synthetic short reads. By way of example, the first long read 606 is divided into first synthetic short reads 702, and the second long read 608 is divided into second synthetic short reads 704. The first synthetic short reads 702 and the second synthetic short reads 704 have expected lengths for short read sequencing data, enabling them to be used by existing short-read variant calling pipelines that are tuned for the short read sequencing data 130. In particular, the first synthetic short reads 702 and the second synthetic short reads 704 preserve their alignment positions from the aligned long reads 512 and may serve as “virtual” short-read alignments that are added to the aligned short reads 510 constructed for the real short reads. By transforming the aligned long reads 512 into a format compatible with short-read pipelines, the techniques described herein enable existing short-read analysis infrastructure to be used while benefiting from the improved coverage of the aligned long reads 512 in difficult to map regions. This compatibility may reduce the need for separate long-read specific analysis pipelines while increasing variant calling accuracy.

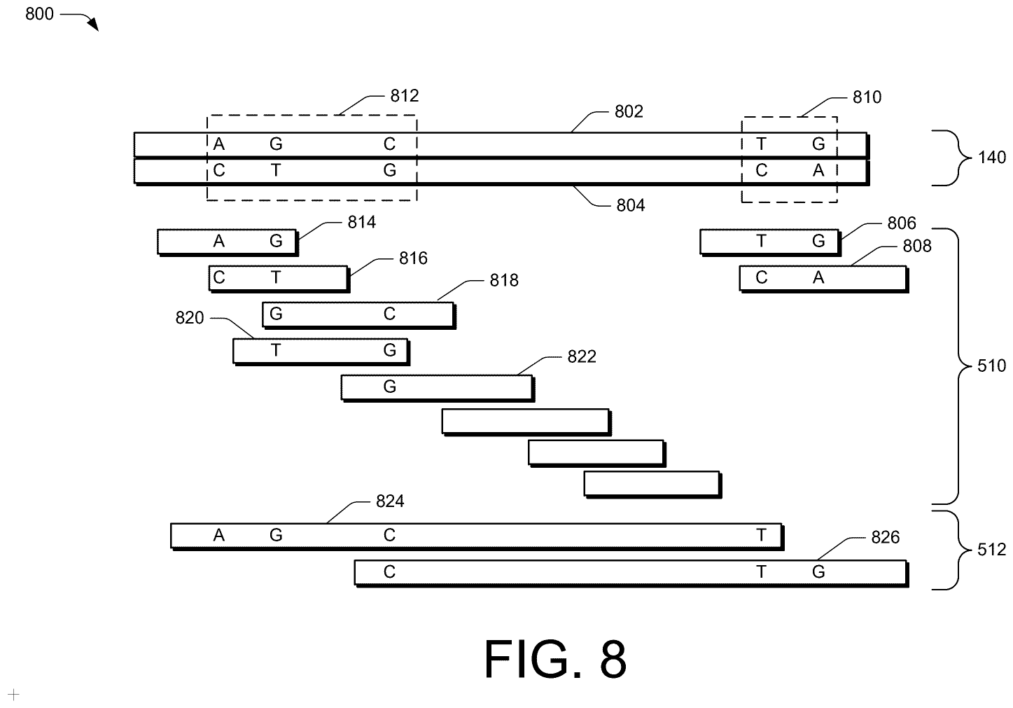

FIG. 8 illustrates an example 800 of integrating short read and long read sequencing to detect phasing. As used herein, “phasing” may refer to the process of determining which genetic variants occur together on the same copy of a chromosome or haplotype. For example, in diploid organisms, which have two copies of each chromosome, phasing may include identifying which alleles or variants are inherited together from each parent. This information may be useful for understanding the relationship between different genetic variations, their potential combined effects, and how they are transmitted across generations. For instance, phasing may provide insights into gene regulation, disease associations, and evolutionary patterns that may not be apparent from unphased genotype data alone.

The example 800 is a simplified example that depicts the aligned short reads 510 and the aligned long reads 512 with respect to the sequence 140. Because the sequence 140 is for a diploid organism, the sequence 140 includes a first genome copy 802 and a second genome copy 804.

The aligned short reads 510 include a first short read 806 and a second short read 808 that span a first sequence region 810 having a first cluster of variants. The first short read 806, for instance, indicates that “T” and “G” occur on the same chromosome (e.g., the first genome copy 802), while the second short read 808 indicates that “C” and “A” occur on the same chromosome (e.g., the second genome copy 804). However, the first short read 806 and the second short read 808 do not include information regarding how these variants relate to a second sequence region 812 having a second cluster of variants.

A third short read 814, a fourth short read 816, a fifth short read 818, a sixth short read 820, and a seventh short read 822 cover the second sequence region 812. However, these reads also do not include information regarding how variants in the second sequence region 812 relate to the first sequence region 810. By way of example, the third short read 814 includes “A” and “G” within the second sequence region 812, but does not relate “A” and “G” in the second sequence region 812 to either “T” and “G” in the first sequence region 810 or “C” and “A” in the first sequence region 810 because the third short read 814 does not span the first sequence region 810. Accordingly, the aligned short reads 510 are not long enough to determine how the variants in the second sequence region 812 relate to the variants in the first sequence region 810.

In the example 800, the aligned long reads 512 include a first long read 824 and a second long read 826. These long reads span longer sections of the sequence 140 compared to the short reads. In particular, the first long read 824 and the second long read 826 include sequence information for both of the first sequence region 810 and the second sequence region 812. For instance, the first long read 824 indicates that “A,” “G,” and “C” within the second sequence region 812 occur on the same chromosome as the “T” within the first sequence region 810. Accordingly, the first long read 824 indicates that these bases occur on the first genome copy 802. As another example, the second long read 826 indicates that “C” within the second sequence region 812 occurs on the same chromosome as “C” and “A” within the first sequence region 810, e.g., on the second genome copy 804.

In this way, the example 800 demonstrates how the combination of short and long read sequencing technologies may be used to determine the phasing of genomic variants. The aligned long reads 512 shown in the example 800 bridge the gap of the aligned short reads 510 between the two clusters of variants, enabling the integrated variant calling module 156 to infer how these variants are organized on the two copies of the genome across much larger distances than traditionally possible with short reads alone. This capability may enhance the accuracy of variant calling and improve the understanding of the phasing of genetic variations in the sequence 140.

Having discussed example details of the techniques for integrated short read and long read sequencing for genomic variant detection, consider now some example procedures to illustrate additional aspects of the techniques.

### Example Procedures

This section describes example procedures for integrated short read and long read sequencing for genomic variant detection in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, the procedures are performed by a suitably configured device, such as the client device 104 and/or the sequencing integrator 106 of FIG. 1.

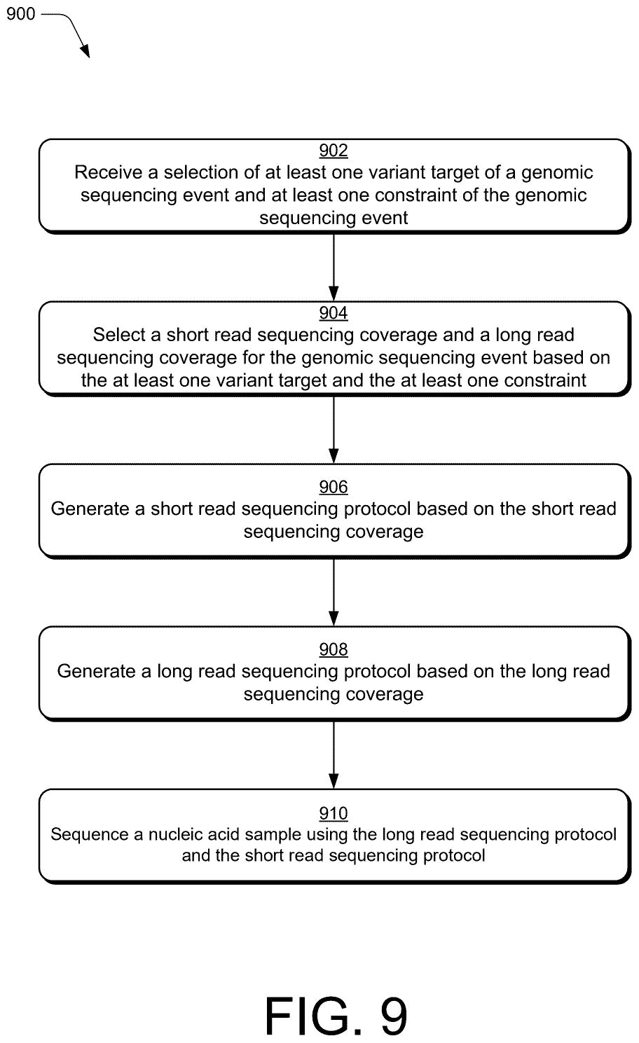

FIG. 9 depicts an example procedure 900 in which sequencing coverage is modulated for a sequencing event.

A selection of at least one variant target of a genomic sequencing event and at least one constraint of the genomic sequencing event is received (block 902). By way of example, the at least one variant target specifies a type of variant that is to be detected via the genomic sequencing event, such as a short variant or a structural variant. Additionally, or alternatively, the at least one variant target further specifies a type of the short variant (e.g., a SNP or an INDEL) or a type of the structural variant (e.g., a CNV, an inversion, or a translocation). It is to be appreciated that more than one type of short variant and/or more than one type of structural variant may be targeted in the genomic sequencing event. Moreover, in at least one variation, another type of sequencing target is used in addition to or as an alternative to the variant target. By way of example, the sequencing target may specify a targeted region of the genome and/or a sequencing technology to be used. The sequencing technology, for instance, defines a short read methodology, sequencer, and/or sequencing center to be used and/or a long read methodology, sequencer, and/or sequencing center to be used.

In accordance with the techniques described herein, the at least one constraint is a performance constraint (e.g., the performance constraint 206). As described above with respect to FIG. 2, the performance constraint indicates one or more desired (e.g., targeted) performance metrics of the genomic sequencing event for performing a variant call. The performance constraint includes one or more or each of an accuracy metric, a sensitivity metric, and a precision metric. Moreover, in at least one implementation, the accuracy metric, the sensitivity metric, and/or the precision metric include a range of values.

Additionally, or alternatively, the at least one constraint is a resource constraint (e.g., the resource constraint 208). The resource constraint is one or both of a monetary cost constraint and a sequencing resource cost constraint. By way of example, the monetary cost is a price per base and/or an overall cost of sequencing a sample. The sequencing resource cost is an amount of data generated by sequencing the sample in the sequencing event 114 and/or a run time of the sequencing event 114, for example. Similar to the performance metrics described above, the resource constraint may include a range of values, such as a price range, a data size range, and/or a sequencing time range.

A short read sequencing coverage and a long read sequencing coverage for the genomic sequencing event are selected based on the at least one variant target and the at least one constraint (block 904). By way of example, a coverage modulation engine (e.g., the coverage modulation engine 120) uses the at least one variant target and the at least one constraint as virtual modulators for selecting the short read sequencing coverage and the long read sequencing coverage. In at least one implementation, the coverage modulation engine generates and utilizes pre-defined relationships between the sequencing coverage and a range of values for the at least one constraint for different combinations of short read sequencing coverages and long read sequencing coverages, such as described above with respect to FIG. 3. The coverage modulation engine references these pre-defined relationships to determine a combination of short read sequencing coverage and long read sequencing coverage that will enable the at least one variant target to be identified within the at least one constraint.

A short read sequencing protocol is generated based on the short read sequencing coverage (block 906). By way of example, a protocol generation module (e.g., the protocol generation module 118) receives the selected short read coverage from the coverage modulation engine and generates the short read sequencing protocol accordingly. The short read protocol, for instance, is usable by a short read sequencer to perform short read sequencing and generate short read sequencing data at the selected short read coverage. The short read sequencing protocol may specify a number of reads to perform, an amount of short read sequencing data to generate, and/or another parameter that is utilized by the short read sequencer in order to generate the short read sequencing data at the selected short read coverage.

In at least one implementation, the protocol generation module generates the short read sequencing protocol by adjusting a previously prepared protocol, such as by adjusting (e.g., increasing or increasing) the number of reads, the amount of short read sequencing data to generate, and/or another parameter. In at least one variation, however, the protocol generation module generates the short read sequencing protocol by determining the short read sequencing protocol without adjusting existing values.

A long read sequencing protocol is generated based on the long read sequencing coverage (block 908). By way of example, the protocol generation module receives the selected long read coverage from the coverage modulation engine and generates the long read sequencing protocol accordingly. The long read protocol, for instance, is usable by a long read sequencer to perform long read sequencing and generate long read sequencing data at the selected long read coverage. For instance, the long read sequencing protocol may specify a number of reads to perform, an amount of long read sequencing data to generate, and/or another parameter that is utilized by the long read sequencer in order to generate the long read sequencing data at the selected long read coverage.

In at least one implementation, the protocol generation module generates the long read sequencing protocol by adjusting a previously prepared protocol, such as by adjusting (e.g., increasing or increasing) the number of reads, the amount of long read sequencing data to generate, and/or another parameter. In at least one variation, however, the protocol generation module generates the long read sequencing protocol by determining the long read sequencing protocol without adjusting existing values.

A nucleic acid sample is sequenced using the long read sequencing protocol and the short read sequencing protocol (block 910). By way of example, the short read sequencer employs a short read sequencing technique, such as next-generation sequencing or Sanger sequencing, to generate the short read sequencing data according to the short read sequencing protocol. Similarly, the long read protocol is usable by the long read sequencer to perform long read sequencing and generate long read sequencing data. The long read sequencer employs a long read sequencing technique, such as single molecule real-time sequencing or nanopore sequencing, to determine the order of nucleotides in each long read and generate the long read sequencing data. In this way, short read and long read sequencing data is generated at customizable short read and long read coverages.

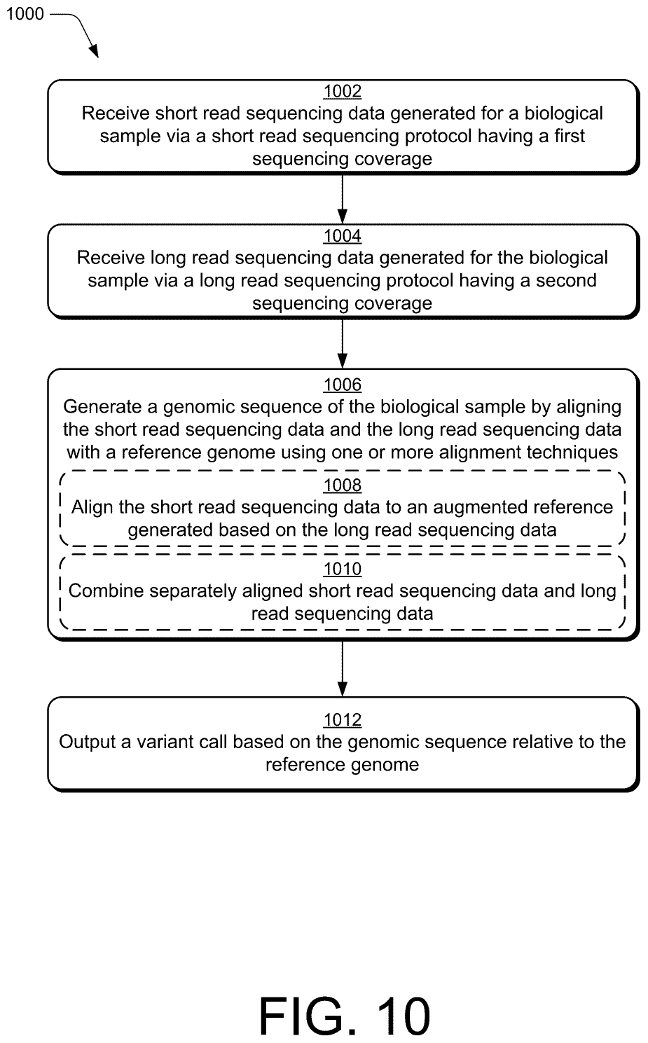

FIG. 10 depicts an example procedure 1000 in which a combination of short read sequencing data and long read sequencing data is used for variant calling. For example, short read sequencing data and the long read sequencing data may be generated according to the procedure 900 of FIG. 9.

Short read sequencing data generated for a biological sample via a short read sequencing protocol having a first sequencing coverage is received (block 1002). By way of example, the short read sequencing data comprises a large collection of short nucleic acid sequences, or short reads, that represent small fragments of a nucleic acid sample derived from the biological sample and sequenced via a genomic sequencing event. A short read, for instance, includes an ordered combination of nucleotides with a length typically ranging from tens to hundreds of bases. In general, a number of short reads and/or an amount of data included in the short read sequencing data decreases as the first sequencing coverage decreases and increases as the first sequencing coverage increases.

Long read sequencing data generated for the biological sample via a long read sequencing protocol having a second sequencing coverage is received (block 1004). By way of example, the long read sequencing data comprises a large collection of longer nucleic acid sequences, or long reads, that represent larger fragments (e.g., in comparison to the short reads) of the nucleic acid sample sequenced via the genomic sequencing event. A long read, for instance, includes an ordered combination of nucleotides with a length typically ranging from thousands to hundreds of thousands of bases. In general, a number of long reads and/or an amount of data included in the long read sequencing data decreases as the second sequencing coverage decreases and increases as the second sequencing coverage increases.

A genomic sequence of the biological sample is generated by aligning the short read sequencing data and the long read sequencing data with a reference genome using one or more alignment techniques (block 1006). By way of example, an alignment module (e.g., the alignment module 138) may employ various approaches to integrate the short read and long read data. As one example, the short read sequencing data is aligned to an augmented reference generated based on the long read sequencing data (block 1008). For instance, the alignment module may align the long reads of the long read sequencing data to the reference genome by using one or more long read alignment algorithms to generate the augmented reference. The augmented reference, for instance, includes sequence differences between the reference genome and an actual sequence of the nucleic acid sample mapped to the reference genome based on sequence portions that are the same between the long reads and the reference genome. Because of their length, the long reads can be mapped to the reference genome with higher confidence than the short reads at positions having larger sequences of variation (e.g., structural variants). The short reads of the short read sequencing data are then mapped, by the alignment module, to the augmented reference via one or more short read alignment algorithms. The aligned short reads and long reads have overlap with respect to each other, which generates a longer, contiguous sequence as the genomic sequence of the biological sample.

As an alternative or in addition, separately aligned short read sequencing data and long read sequencing data are combined (block 1010). By way of example, the alignment module may separately align both short and long reads to the reference genome and then combine the alignments to generate a consensus sequence and/or a hybrid alignment. In at least one implementation, the long read sequencing data is divided (e.g., fragmented) into synthetic short reads after it is aligned, which enables the long read sequencing data to be efficiently used in existing short-read variant calling pipelines. Because of their length, the long reads can be mapped to the reference genome with higher confidence than the short reads at positions having larger sequences of variation (e.g., structural variants), and this information is leveraged in the integration process to improve the overall alignment accuracy and contiguity of the resulting genomic sequence. By way of example, the hybrid alignment may be generated by combining the aligned synthetic short reads with a short read alignment of the short read sequencing data.

A variant call is output based on the genomic sequence relative to the reference genome (block 1012). By way of example, an integrated variant calling module (e.g., the integrated variant calling module 156) compares the genomic sequence and/or alignment (e.g., the short read alignment, the long read alignment, and/or the hybrid alignment) with the reference genome and identifies positions where the genomic sequence differs from the reference genome. In accordance with the techniques described herein, the integrated variant calling module uses one or more short variant calling algorithms to identify short variants (e.g., SNPs or INDELs) and one or more structural variant calling algorithms to identify structural variants (e.g., CNVs, inversions, translocations, large deletions, and so forth). The integrated variant calling module further uses one or more refinement algorithms to validate and/or correct the short variants identified by the one or more short variant calling algorithms based on the one or more structural variant calling algorithms, or the output thereof. Similarly, the integrated variant calling module uses the one or more refinement algorithms to validate and/or correct the structural variants identified by the one or more structural variant calling algorithms based on the one or more short variant calling algorithms, or the output thereof. In this way, the short read sequencing data and long read sequencing data is combined for resource-efficient and effective variant calling.

In at least one implementation, the integrated variant calling module evaluates the short read sequencing data and the long read sequencing data in separate variant calling pipelines. Alternatively, the short read sequencing data and the long read sequencing data are evaluated in a single variant calling pipeline, such as when the long read sequencing data is divided into synthetic short reads to generate the hybrid alignment. Accordingly, the techniques described herein enable the short read sequencing data and the long read sequencing data to be combined in various ways that enable both short and structural variant calling with high accuracy and precision and reduced sequencing coverage compared to when short read sequencing and long read sequencing are performed alone.

The integrated variant calling module may further provide genotype-aware statistics and/or phasing information. By leveraging both short and long read data, the module can determine not only the presence of variants but also their zygosity (heterozygous or homozygous) and phase relationship. The long reads, with their ability to span multiple variant sites, provide information for phasing variants across longer genomic distances. This phasing information can be used to construct haplotypes, which represent the specific combinations of alleles that occur together on the same chromosome. The short reads, with their higher depth of coverage, may contribute to more accurate genotyping of individual variants. By combining these complementary strengths, the integrated variant calling module may produce a more comprehensive view of the genome, including genotype-aware variant calls and phased haplotypes. This enhanced information may provide information regarding the functional impact of variants, especially in cases where multiple variants may interact or where the specific combination of variants on each chromosome is clinically relevant. The ability to provide such detailed genetic information in a resource-efficient manner represents a significant advancement in genomic analysis capabilities.

Having described example procedures in accordance with one or more implementations, consider now an example system and device that can be utilized to implement the various techniques described herein.

### Example System and Device

FIG. 11 illustrates an example system generally at 1100 that includes an example computing device 1102 that is representative of one or more computing systems and/or devices that may implement the various techniques described herein. This is illustrated through inclusion of the sequencing integrator 106. The computing device 1102 may be, for example, a server of a service provider, a device associated with a client (e.g., a client device), an on-chip system, and/or any other suitable computing device or computing system.

The example computing device 1102 as illustrated includes a processing system 1104, one or more computer-readable media 1106, and one or more I/O interfaces 1108 that are communicatively coupled, one to another. Although not shown, the computing device 1102 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionality to perform one or more operations using hardware. Accordingly, the processing system 1104 is illustrated as including hardware elements 1110 that may be configured as processors, functional blocks, and so forth. This may include implementation in hardware as an application specific integrated circuit or other logic device formed using one or more semiconductors. The hardware elements 1110 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 1106 is illustrated as including memory/storage 1112. The memory/storage 1112 represents memory/storage capacity associated with one or more computer-readable media. The memory/storage 1112 may include volatile media (such as random-access memory (RAM)) and/or nonvolatile media (such as read only memory (ROM), Flash memory, optical disks, magnetic disks, and so forth). The memory/storage 1112 may include fixed media (e.g., RAM, ROM, a fixed hard drive, and so on) as well as removable media (e.g., flash memory, a removable hard drive, an optical disc, and so forth). The computer-readable media 1106 may be configured in a variety of other ways as further described below.

Input/output interface(s) 1108 are representative of functionality to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1102. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1110. The computing device 1102 may be configured to implement particular instructions and/or functions corresponding to the software and/or hardware modules. Accordingly, implementation of a module that is executable by the computing device 1102 as software may be achieved at least partially in hardware, e.g., through use of computer-readable storage media and/or hardware elements 1110 of the processing system 1104. The instructions and/or functions may be executable/operable by one or more articles of manufacture (for example, one or more computing devices 1102 and/or processing systems 1104) to implement techniques, modules, and examples described herein.

The techniques described herein may be supported by various configurations of the computing device 1102 and are not limited to the specific examples of the techniques described herein. This functionality may also be implemented all or in part through use of a distributed system, such as over a “cloud” 1114 via a platform 1116 as described below.

The cloud 1114 includes and/or is representative of a platform 1116 for resources 1118, which are depicted including the sequencing integrator 106. The platform 1116 abstracts underlying functionality of hardware (e.g., servers) and software resources of the cloud 1114. The resources 1118 may include applications and/or data that can be utilized while computer processing is executed on servers that are remote from the computing device 1102. Resources 1118 can also include services provided over the Internet and/or through a subscriber network, such as a cellular or Wi-Fi network.

The platform 1116 may abstract resources and functions to connect the computing device 1102 with other computing devices. The platform 1116 may also serve to abstract scaling of resources to provide a corresponding level of scale to encountered demand for the resources 1118 that are implemented via the platform 1116. Accordingly, in an interconnected device embodiment, implementation of functionality described herein may be distributed throughout the system 1100. For example, the functionality may be implemented in part on the computing device 1102 as well as via the platform 1116 that abstracts the functionality of the cloud 1114.

Having discussed example details of the techniques for integrated short read and long read sequencing for genomic variant detection, consider now an example to illustrate usage of the techniques.

Example Application: Blended Length Genome Sequencing (Blend-Seq): Combining Short Reads with Low-Coverage Long Reads to Maximize Variant Discovery

Blend-seq is introduced as a method for combining data from traditional short-read sequencing pipelines with low-coverage long reads, with the goal of substantially improving variant discovery for single samples without the full cost of high-coverage long reads. It is demonstrated that by using 4× long read coverage to augment 30× short read coverage, SNP discovery across the genome is improved. Moreover, precision and recall beyond what is possible with short reads is achieved, even at very high short read coverage (60×). For genotype-agnostic discovery of structural variants, a threefold improvement in recall while maintaining precision is observed by using the low-coverage long reads on their own. For the more specialized scenario of genotype-aware structural variant calling, combining the long and short reads in a graph-based approach results in greater performance than either technology on its own. The observed gains highlight the complementary nature of short and long read technologies: long reads help with SNP discovery by better mapping to difficult regions, and they provide better performance with long insertions and deletions (structural variants) by virtue of their length, while the larger number of short-read layers help with genotyping structural variants discovered by long reads. In this way, blend-seq offers many of the benefits of long-read pipelines without incurring the cost of high-coverage long reads.

While market-leading short-read sequencing technologies are excellent at capturing small variations (SNPs and INDELs) in most of the human genome, they are far less effective both for hard-to-map regions as well as for structural variants (SVs). Long-read technologies, on the other hand, have shown remarkable success in both of these areas; however, the significantly higher cost has left them out of reach for many applications. Blended length genome sequencing (blend-seq) is introduced herein to combine traditional short-read pipelines with very low coverage long reads, demonstrating how substantial gains in both short and long variant calling for single samples can be made without the full cost of high- or medium-coverage long-read sequencing.

Blend-seq is flexible with respect to choices and coverage levels of each sequencing technology, and the methods and choices described herein can be adapted to a wide variety of setups. A pragmatic setup for existing practices is to augment the typical 30× coverage of short reads with 4× coverage of long reads. For short variants, it is shown that with this setting, read types can be combined to improve SNP recall (while maintaining precision)—not only in hard-to-map regions, but across the entire genome, even compared to very high coverage short-read performance, which levels off well before 60×. For SVs, it is shown that recall can be improved by a factor of three across the genome, and by even more for variants overlapping with the exome, by using the low coverage long reads on their own. For settings where the genotype of structural variants matter, even greater gains are shown by combining short and long reads in a graph-based approach.

The implications for the SNP results are immediate: the vast majority of known genetic diseases are identified by these single nucleotide polymorphisms, and so improvements to SNP recall have the potential to impact clinical diagnosis. Such gains apply to the discovery of new variant associations as well: greater recall implies a greater ability to associate SNPs with phenotypic profiles in biobank or cohort data. As such, this improvement in SNP performance has the potential of both improving existing diagnostic procedures and the discovery of new diagnostics.

At the same time, SNPs do not provide a full indication of genetic variation. From population genomics to understanding disease mechanics, there is a need to move beyond short variants and consider a wider set of causes for genetic disease. To date, the most informative way to address these longer variants has been through long-read technologies, such as those from Pacific Biosciences (PacBio), with reads of 15-20 kilobases (kb), and Oxford Nanopore Technologies (ONT), with reads of up to 2 megabases (mb). These far longer reads can span much larger variants, while simultaneously mapping more accurately to the correct locations in the human genome, particularly in low-complexity regions.

The hypothesis of the Example Application is that even a small amount of long-read information could greatly augment short-read results, shedding significant additional light on these problems without incurring the full cost of high-depth long-read sequencing. More generally, the complementary capabilities of varying levels of short and long reads could be balanced to best tune their joint performance for a specific application. The blend-seq approach presented herein thus includes varying the choice and coverage for each technology, leveraging their individual and joint capabilities to provide gains in short variants, structural variant discovery, and structural variant genotyping. Results are shown using PacBio and ONT technologies for long reads, coupled with Illumina sequencing for short reads, all with varying levels of coverage. Moreover, different choices lead to differential performance gains for specific regions and classes of variants. This tuning approach provides a continuous path to increasing the amount and quality of the desired genomic information per unit of cost, modulated by the “control knobs” of levels of coverage as well as the choices of specific short/long read technologies.

These gains are demonstrated with three approaches, varying in complexity and degree of integration between short- and long-read pipelines. For improvements in SNP discovery, the mapping capabilities of low-coverage long reads are leveraged, then these reads are broken up into “virtual” short reads that can be combined with true short reads in a state-of-the-art Dynamic Read Analysis for GENomics (DRAGEN) calling pipeline. For SV discovery, the low-coverage long reads are simply used to call structural variants without assistance from the short reads. For SV genotyping, a sample-specific graph reference is created that augments the standard reference with information from the long reads, then the short reads are mapped to this augmented reference for calling genotype-aware structural variants.

There have been some prior investigations on combining short and long reads. However, the Example Application described herein is focused on the specific scenario of combining standard coverage short reads with low coverage long reads, and doing so using independent, unmodified pipelines using only in silico methods, which is different than what has been previously explored. Prior work has instead either required high coverage for both long and short reads or required library preparation changes to establish biochemical correspondence between the long and short reads. For example, one existing technique uses high coverage long reads (12-15×) in addition to standard short read coverage to improve variant discovery for diagnostic scenarios. Beyond this, the first category (high coverage long and short reads) is dominated by methods for hybrid de novo assembly, in which long and short reads are integrated to improve assembly quality. The second category (library preparation changes) includes a wide variety of approaches, including read clouds, synthetic long reads, and linked reads as mechanisms to improve assembly. In these approaches, the correspondence between short and long molecules is established in vitro. Finally, since the focus is on performance with real data, simulated read mixes are not investigated or compared herein.

### Results

Results are described in three areas detailed in the subsections below. The first is on short variants and shows how modifying short-read calling pipelines to leverage long-read information along with short reads can result in significant gains in SNP recall across the genome, and particularly in hard-to-map regions. The second section focuses on structural variants and shows how the recall of SVs across the genome can be increased by a factor of three with just a few layers of long reads. The last section investigates the setting where genotyping is useful for SVs and shows how a graph-based approach can combine long and short reads to provide genotype-aware SV calling performance that exceeds either technology in isolation.

### Short Variant Calling Performance

In this first subsection, improvements to the calling of short variants (e.g., events less than 50 bp in length) with blend-seq are described. First, it is shown that SNP recall can be substantially improved (while maintaining precision) across the genome and by an even greater amount in hard-to-map regions. This is achieved by breaking mapped long reads into synthetic short reads that can augment existing short reads in the DRAGEN pipeline. Next, the sources of these gains are investigated to understand what fractions are coming from hard-to-map regions versus other scenarios. Finally, the same approach for INDELs is examined, and it is found that while some gains in recall can be achieved, the effect is diluted by a drop in precision.

### Approach and Data

While short-read pipelines perform well in regions where their read lengths are sufficient to map unambiguously to the genome, their performance suffers when the mapping is no longer unique; conversely, long reads are better at mapping uniquely to these more difficult regions. Thus, the approach described herein sought to leverage the higher-quality mapping of long reads within a state-of-the-art short-read calling pipeline. The approach was to map the long reads on their own, then break each alignment into segments of short-read size. These “virtual” short-read alignments were then passed into the short-read calling pipeline along with the alignments of the actual short reads, to create a hybrid BAM, which was then used for variant calling.

Experiments were performed with varying levels of short-read coverage, from 10× to 60×, simulated by downsampling a single set of reads (Illumina NovaSeq X) for a single sample (human sample HG002). The Illumina reads were processed using DRAGEN v3.7.8, an Illumina short-read pipeline employed in several large-scale genomic projects. In particular, DRAGMAP was used to align the reads.

For the long reads, four layers (4×) of PacBio Revio long reads were used. This level of coverage was selected based on the tradeoffs between coverage and discovery performance for structural variants described further below. The analysis involved first mapping the long reads using minimap2, then partitioning these alignments into disjoint chunks of length 151 base pairs (bp) to match the expectations of DRAGEN for short reads. The alignments preserved their original positions and CIGAR strings. These “virtual” short-read alignments were then added to the BAM constructed for the real short reads. DRAGEN v3.7.8 was then used to call variants from this hybrid BAM.

The results of the short reads alone (with DRAGEN), the 4× long reads alone (with DeepVariant), and the hybrid regime described above, were compared against the NIST truth dataset for HG002 over their high-confidence interval regions. In addition to calculating whole-genome precision and recall statistics, these were also collected over some subregions of interest as determined by NIST.

### SNP Performance

Using the hybrid method, SNP recall with blend-seq showed significant improvements over short reads alone (at any coverage), while maintaining precision, for combinations with both PacBio and ONT. The peak performance of blend-seq is with 25-30× range of short-read coverage, where it significantly outperforms short reads alone in terms of recall while matching precision, even when compared to very high levels of short-read coverage where short-read performance appears to level off (see Table 1 for detailed values). Even at relatively low levels of short-read coverage (12×), recall exceeding this maximal short-read recall was observed, despite a small loss in precision in ONT (Table 1).

TABLE 1
SNP Performance by Region

| | WholeGenome | | | LowMappability | | | Exome | | |
|---|---|---|---|---|---|---|---|---|---|
| | | | F1 | | | F1 | | | F1 |
| | Recall | Precision | Score | Recall | Precision | Score | Recall | Precision | Score |
| IL | .9889 | .9944 | .9916 | .8556 | .9312 | .8918 | .9857 | .9870 | .9864 |
| 15X | | | | | | | | | |
| control | | | | | | | | | |
| IL | .9926 | .9961 | .9944 | .8813 | .9461 | .9125 | .9887 | .9902 | .9894 |
| 30X | | | | | | | | | |
| control | | | | | | | | | |
| IL | .9929 | .9963 | .9946 | .8856 | .9490 | .9162 | .9895 | .9901 | .9898 |
| 60X | | | | | | | | | |
| (high | | | | | | | | | |
| depth) | | | | | | | | | |
| blend- | .9956 | .9927 | .9942 | .9449 | .9178 | .9311 | .9927 | .9864 | .9895 |
| seq | | | | | | | | | |
| 15X | | | | | | | | | |
| IL + | | | | | | | | | |
| 4X | | | | | | | | | |
| ONT | | | | | | | | | |
| blend- | .9956 | .9951 | .9954 | .9409 | .9439 | .9424 | .9934 | .9895 | .9914 |
| seq | | | | | | | | | |
| 15X | | | | | | | | | |
| IL + | | | | | | | | | |
| 4X PB | | | | | | | | | |
| blend- | .9965 | .9952 | .9958 | .9462 | .9382 | .9422 | .9933 | .9892 | .9912 |
| seq | | | | | | | | | |
| 30X | | | | | | | | | |
| IL + | | | | | | | | | |
| 4X | | | | | | | | | |
| ONT | | | | | | | | | |
| blend- | .9963 | .9964 | .9963 | .9413 | .9521 | .9467 | .9935 | .9910 | .9922 |
| seq | | | | | | | | | |
| 30X | | | | | | | | | |
| IL + | | | | | | | | | |
| 4X PB | | | | | | | | | |
| 30X | .9989 | .9993 | .9991 | .9861 | .9944 | .9902 | .9983 | .9991 | .9987 |
| PB | | | | | | | | | |
| (high | | | | | | | | | |
| depth) | | | | | | | | | |

An even stronger improvement in performance was observed in regions that are hard to map for short reads, substantially improving recall compared to short reads alone while making small gains in precision for the PacBio blend (see Table 1 for detailed values). These regions are difficult due to the limitations of short reads; as such, long reads were expected to have less difficulty mapping unambiguously. Even with only four layers of long-read coverage, the complementary nature of the short and long reads boosting performance can be seen. While improvements across the genome and in difficult regions were promising, these gains might only appear in regions of the genome where variants would have low interpretability. To investigate whether this was the case, the analysis was run restricted to the exome. The result is that a similar gain in recall as seen for the whole genome is observed even in these more interpretable regions (see Table 1 for details). Since most SNPs used for clinical diagnosis are in the exome, this increases the potential relevance of these improvements in diagnostic settings.

### Sources of SNP Improvement

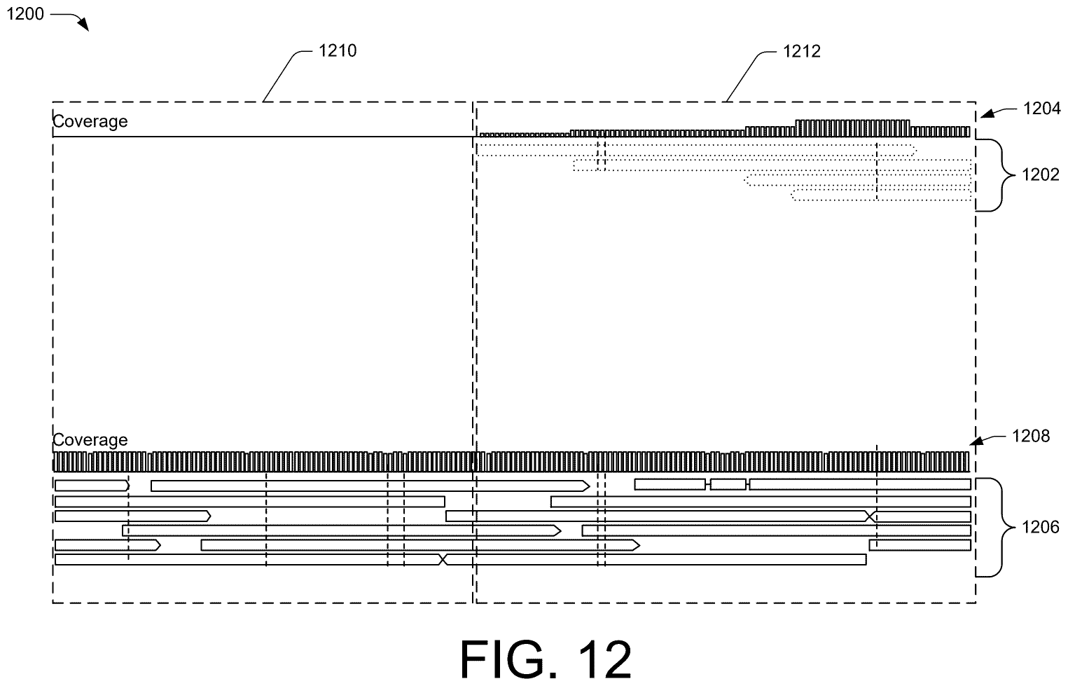

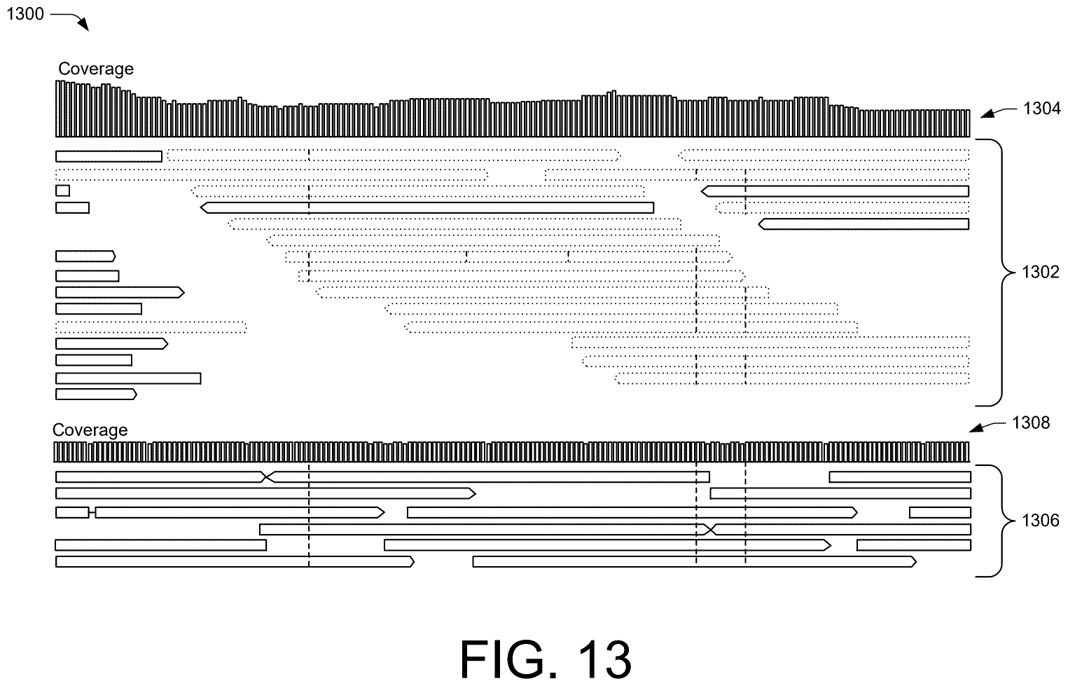

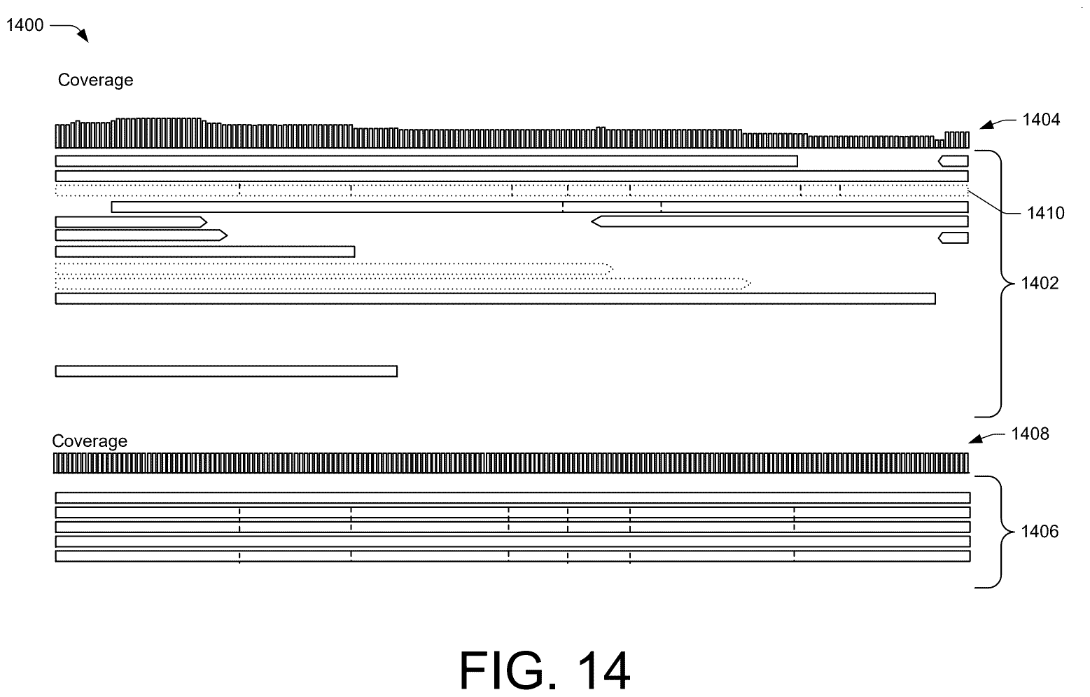

To better understand the source of this boost in SNP performance, a detailed analysis was performed into characterizing the new SNPs discovered using the hybrid readset. The focus was on the low-mappability regions, since these experienced the largest gains. In particular, the additional SNPs captured via this approach were examined. These SNPs were previously labeled false negatives with short reads alone but became true positives with the hybrid method. After restricting the data to these cases, a subset of sites was manually inspected with the Integrative Genomics Viewer (IGV). Cases (see, e.g., FIG. 12) of SNPs were observed where the short-read support either had zero depth or all reads had mapping quality zero. In both cases, the variant caller would have no evidence to use when making a call. Situations (see, e.g., FIG. 13) were also observed where nearly all short reads have mapping quality zero. In these situations the short reads are disadvantaged from making a call, but not deterministically across runs. Other examples (see, e.g., FIG. 14) show a more complex situation where reference-supporting reads map well, but the few reads representing a correct haplotype within a cluster of nearby SNPs have mapping quality zero.

FIG. 12 shows a first example alignment 1200 of hybrid short read and long read sequencing data. The first example alignment 1200 includes aligned short reads 1202 having a short read coverage 1204 and aligned fragmented long reads 1206 having a long read coverage 1208. Vertical dashed lines indicate the sites of SNPs detected in a given read. Reads having a dotted outline have poor mapping quality (e.g., mapping quality zero), while reads having a solid outline have good mapping quality.

In the first example alignment 1200, the aligned short reads 1202 have poor mapping quality, whereas the aligned fragmented long reads 1206 have good mapping quality. The aligned short reads 1202 have zero depth in a first region 1210, as indicated by the lack of short reads as well as the short read coverage 1204. The aligned fragmented long reads 1206 have good long read coverage 1208 in the first region 1210 and are usable to identify SNPs within the first region 1210. Moreover, although the aligned short reads 1202 indicate the SNPs within a second region 1212, the poor mappability would cause the variant caller to disregard this evidence. Accordingly, the aligned fragmented long reads 1206 further provide evidence for the SNPs in the second region 1212 due to the good mapping quality of the aligned fragmented long reads 1206.

FIG. 13 shows a second example alignment 1300 of hybrid short read and long read sequencing data. The second example alignment 1300 includes aligned short reads 1302 having a short read coverage 1304 and aligned fragmented long reads 1306 having a long read coverage 1308. Vertical dashed lines indicate the sites of SNPs detected in a given read. Reads having a dotted outline have poor mapping quality (e.g., mapping quality zero), while reads having a solid outline have good mapping quality.

In the second example alignment 1300, the aligned short reads 1302 have mostly poor mapping quality (e.g., mapping quality zero), whereas the aligned fragmented long reads 1306 have good mapping quality. As such, even though the short read coverage 1304 is sufficient, the poor mapping quality means that the aligned short reads 1302 do not provide sufficient evidence for calling the SNPs. Accordingly, the aligned fragmented long reads 1306 provide most of the evidence to the variant callers for the SNPs indicated in the second example alignment 1300.

FIG. 14 shows a third example alignment 1400 of hybrid short read and long read sequencing data. The third example alignment 1400 includes aligned short reads 1402 having a short read coverage 1404 and aligned fragmented long reads 1406 having a long read coverage 1408. Vertical dashed lines indicate the sites of SNPs detected in a given read. Reads having a dotted outline have poor mapping quality (e.g., mapping quality zero), while reads having a solid outline have good mapping quality.

In the third example alignment 1400, the aligned short reads 1402 are split between poor mapping quality (e.g., mapping quality zero) and good mapping quality and provide evidence for both the SNP and the reference allele. In particular, a short read 1410 provides support for the SNPs but has poor mapping quality, e.g., due to low mappability of the genomic region as the higher concentration of SNPs in this region. As such, even though the aligned short reads 1302 have good short read coverage 1304, the aligned short reads 1302 do not provide SNP-supporting evidence to the variant caller. The aligned fragmented long reads 1406 have good mapping quality and thus provide support for the SNPs to the variant caller.

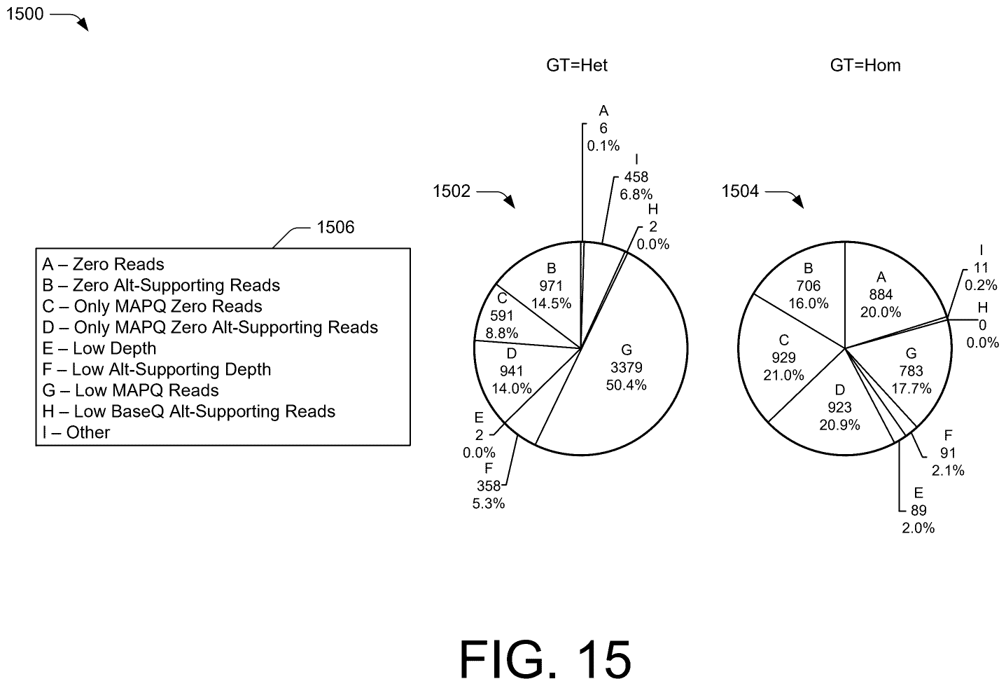

To better understand how general these cases are, categories were assigned to these additional SNPs gained by the method in the low-mappability regions. FIG. 15 shows an example classification 1500 of new SNPs identified using a hybrid readset of short read and long read sequencing data. The classification 1500 includes a first pie chart 1502 for heterozygous sites and a second pie chart 1504 for homozygous sites. Portions are of the first pie chart 1502 and the second pie chart 1504 are labeled according to the category of evidence supporting them, as indicated by a legend 1506. The category labels, based on mapping depth and quality, articulate the different reasons why a SNP was missed in the pure short-read data but captured in the hybrid regime (details are provided in the Methods section). The breakdown of these labels for the SNPs in question is given by the pie charts in FIG. 15 for the 30× short-read and 4× long-read mix (30× mix experiment).

The first four categories (Zero Reads (A), Zero Alt-Supporting Reads (B), Only MAPQ Zero Reads (C), Only MAPQ Zero Alt-Supporting Reads (D)) represent the case where the pure short read variant caller has no usable evidence to make the correct call. In these categories, the long reads were able to add valuable supporting haplotypes-enough to salvage the site and make the correct call. In the 30× mix experiment, about 37% of heterozygous sites (the first pie chart 1502) and about 78% of homozygous sites (the second pie chart 1504) among those “corrected” in the long reads mix fall into these categories. The remaining categories (Low Depth (E), Low Alt-Supporting Depth (F), Low MAPQ Reads (G), Low BaseQ Alt-Supporting Reads (H), and Other (I)) are mostly sites where the pure short-read evidence is weak but not absent. It was observed that in other levels of short-read coverage (e.g., 20× and 10×), there is a similar fraction of gained SNPs coming from the first four categories, with long-read evidence added to pileups where pure short reads had no evidence.

### INDEL Performance

INDELs are defined as insertion or deletion events of length less than 50 bp. Applying the hybrid method described above over the entire genome, it was found that INDEL performance has greater recall for the PacBio blend compared to short reads alone, but precision is marginally lower without any additional filtering. There was a greater boost in recall over low-mappability regions, but the overall effect was diluted by a lower precision. This is most likely due to a higher INDEL error rate in long read alignments and the fact that current tools are tuned to Illumina error rates, driving false positives for this variant type. See Table 2 for details.

TABLE 2
INDEL Performance by Region

| | WholeGenome | | | LowMappability | | | Exome | | |
|---|---|---|---|---|---|---|---|---|---|
| | | | F1 | | | F1 | | | F1 |
| | Recall | Precision | Score | Recall | Precision | Score | Recall | Precision | Score |
| IL | .9826 | .9900 | .9863 | .8258 | .9168 | .8689 | .9813 | .9659 | .9735 |
| 15X | | | | | | | | | |
| control | | | | | | | | | |
| IL | .9946 | .9964 | .9955 | .8593 | .9354 | .8957 | .9938 | .9779 | .9858 |
| 30X | | | | | | | | | |
| control | | | | | | | | | |
| IL | .9959 | .9972 | .9965 | .8647 | .9364 | .8991 | .9958 | .9780 | .9868 |
| 60X | | | | | | | | | |
| high | | | | | | | | | |
| depth | | | | | | | | | |
| blend- | .9588 | .8173 | .8824 | .9171 | .5952 | .7218 | .9875 | .4681 | .6351 |
| seq | | | | | | | | | |
| 15X | | | | | | | | | |
| IL + | | | | | | | | | |
| 4X | | | | | | | | | |
| ONT | | | | | | | | | |
| blend- | .9861 | .9643 | .9751 | .9197 | .8368 | .8763 | .9917 | .8602 | .9213 |
| seq | | | | | | | | | |
| 15X | | | | | | | | | |
| IL + | | | | | | | | | |
| 4X PB | | | | | | | | | |
| blend- | .9932 | .9811 | .9871 | .9248 | .7253 | .8130 | .9938 | .8665 | .9258 |
| seq | | | | | | | | | |
| 30X | | | | | | | | | |
| IL + | | | | | | | | | |
| 4X | | | | | | | | | |
| ONT | | | | | | | | | |
| blend- | .9965 | .9949 | .9957 | .9241 | .8889 | .9062 | .9938 | .9625 | .9779 |
| seq | | | | | | | | | |
| 30X | | | | | | | | | |
| IL + | | | | | | | | | |
| 4X PB | | | | | | | | | |
| 30X | .9845 | .9776 | .9810 | .9811 | .9869 | .9840 | .9979 | .9273 | .9613 |
| PB | | | | | | | | | |
| high | | | | | | | | | |
| depth | | | | | | | | | |

### Structural Variant Discovery

In this subsection, the use of blend-seq at low long-read coverage for SV discovery is investigated. This is motivated by the well-established advantage of long reads for structural variant detection as compared to short reads. In this first scenario, the focus is on retrieving SVs without concern for their underlying genotype; genotype-aware retrieval is considered in the next subsection. Unlike the earlier results for SNPs, the best performance in this setting comes from using low-coverage long reads alone with conventional long-read calling pipelines; the short reads are not utilized at all. However, it is to be appreciated that in at least one variation, the short reads are utilized in addition to the low-coverage long reads or as an alternative to the low-coverage long reads.

Results are first shown over the entire genome, demonstrating an over three-fold increase in SV recall while maintaining precision. The results are then broken down in two ways: first, to investigate where the gains are coming from, SV performance is studied by genomic regions, including regions overlapping the exome, NIST-defined difficult regions (those that are harder to map for short reads), and easier regions (the complement of difficult regions). Second, the results are examined across SV types, to see what classes of SVs are most improved by this approach.

### Approach and Data

The performance of SV detection at different levels of long-read coverage was investigated, focusing on structural variants (large insertions and deletions with length ≥50 bp) in the HG002 sample discovered using Sniffles2 and allowing even a single read of evidence to support a call (results from other state-of-the-art SV callers displayed similar trends and are omitted for brevity). The outputs were cleaned and benchmarked with Truvari against the NIST-Q100 SV V1.0 truth, a set of calls derived from the highest-quality long-read assembly of HG002 available to date. Default values were used for all parameters of Truvari other than sequence comparison, which was turned off. This latter setting was used to fairly compare the benchmarking results with the pure short-read pipeline, which does not report inserted bases. Note that with default settings Truvari, labels variants as true positive, false positive, etc. based solely on matching alleles (up to some ambiguity in position, length, etc.) and does not consider the genotype by default; genotyping accuracy is considered in more detail in the following section.

For the long reads, PacBio Revio reads and ONT R10.4 reads from a public experiment, downsampled to 1-10×, were used as experimental groups, and the full 30× setting was also included to examine high-coverage long read performance. These were compared against the “GATK-SV callset,” which includes the HG002-specific calls from a recent Illumina 30×cohort-level callset. This callset is the result of performing multi-sample SV calling with GATK-SV and subsequently filtering by several criteria (including restricting results to HG002). GATK-SV is a state-of-the-art ensemble short-read SV discovery pipeline that integrates the output of several specialized callers, including Manta (developed by Illumina), cn.MOPS and GATK-gCNV for copy-number variants, and MELT for mobile element insertions. Note that unlike the method described herein, which operates on individual samples in isolation, GATK-SV can only be run on cohorts, as it leverages information from multiple samples to improve its precision.

A few remarks are provided with respect to prior investigation in this area. First, long read downsampling analyses for SV discovery have been performed in the past, but those studies either did not consider coverage levels as low as those presented here, used more stringent parameters in the callers, or compared to less accurate and complete truth sets. Second, although it is in principle possible to model the theoretical recall of structural variants as a function of coverage and read length, practical issues like the association of SVs with repeats and the effect of sequencing errors and of read alignment algorithm on SV calling rely on the systematic empirical investigation presented here. Next, for the PacBio reads, it is noted that the sniffles2-based long read SV recall is lower than what is reported elsewhere, even for high levels of coverage. This is because phasing information was not available for the hybrid data, as is further explained in the Discussion below. Finally, for the ONT reads, which are known to have lower base quality, prior work has shown that short reads can be used to ameliorate this issue.

### SV Performance by Genomic Region

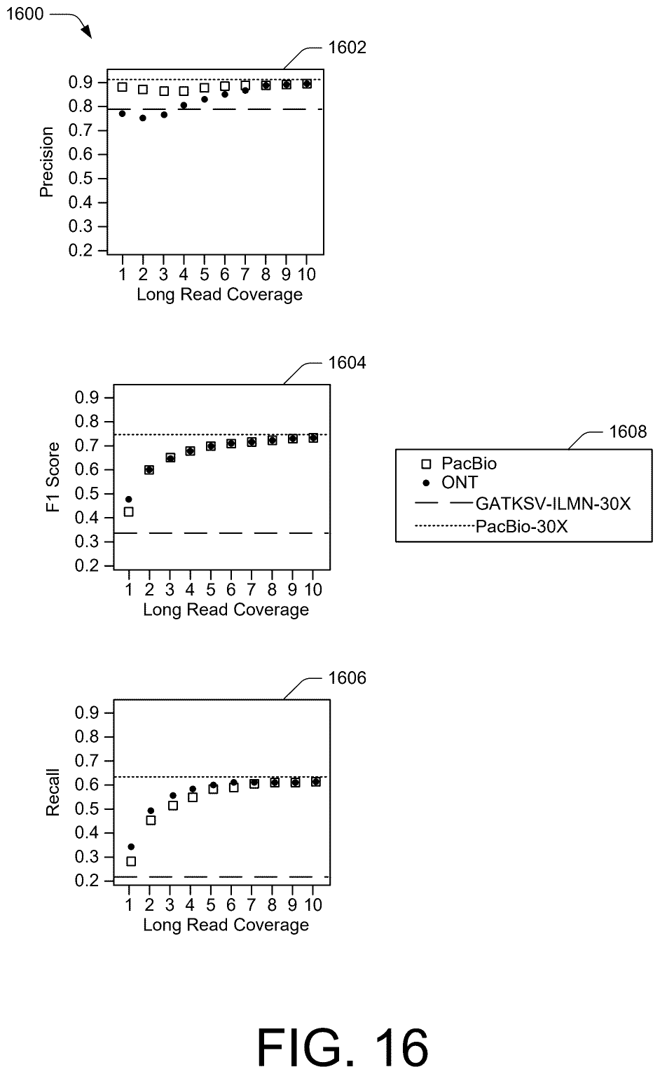

FIG. 16 shows an example 1600 of structural variant discovery with long reads versus short reads. The example 1600 includes a first graph 1602 of precision (vertical axis) versus long read coverage (horizontal axis), a second graph 1604 of F1 score (vertical axis) versus long read coverage (horizontal axis), and a third graph 1606 of recall (vertical axis) versus long read coverage (horizontal axis). A legend 1608 indicates that long read data obtained via PacBio is indicated by white (or open-filled) squares, long read data obtained via ONT is indicated by black circles, the GATK-SV callset is indicated by long-dashed lines, and 30×long-read performance is indicated by short-dashed lines.

As shown in the example 1600, even at one layer of coverage, the long reads outperformed the short reads in terms of precision (the first graph 1602) and recall (the second graph 1604) for the genome as a whole. With four layers of coverage, long reads substantially outperformed short reads in all regions. Diminishing returns on long-read depth beyond a small number of layers were observed, with results approaching the 30× long-read performance at 10× coverage.

At 4× coverage for both PacBio and ONT, much of the gap to the full 30× coverage is closed using this pipeline; as such, this serves as a compelling coverage/performance tradeoff. The results were broken down across regions of interest, such as the exome, difficult regions (NIST's “AllDifficult” regions, deemed to be difficult for sequencing by short-read technologies), and easy regions (the complement of difficult regions). Table 3 shows details for all of these regions for short reads alone, 1× and 4× coverage for both PacBio and ONT, and 30× PacBio (a proxy for the asymptotic limit of this technology). Similar trends are seen in each set of regions, with 4× long reads substantially outperforming the short reads even in the easier regions where short reads are expected to perform the best.

TABLE 3
SV Discovery by Region

| | Whole Genome | | | Easy Regions | | | Hard Regions | | | Exome | | |
|---|---|---|---|---|---|---|---|---|---|---|---|---|
| | | | F1 | | | F1 | | | F1 | | | F1 |
| | Recall | Precision | Score | Recall | Precision | Score | Recall | Precision | Score | Recall | Precision | Score |
| IL 30X | .2126 | .7830 | .3344 | .4030 | .8606 | .5490 | .1759 | .7576 | .2855 | .1096 | .5147 | .1807 |
| control | | | | | | | | | | | | |
| ONT 1X | .3454 | .7688 | .4767 | .4109 | .7276 | .5252 | .3381 | .8215 | .4790 | .3455 | .7741 | .4778 |
| PacBio 1x | .2742 | .8822 | .4183 | .3226 | .9345 | .4796 | .2655 | .8780 | .4077 | .2070 | .8354 | .3318 |
| ONT 4X | .5815 | .7987 | .6730 | .6932 | .7830 | .7354 | .5675 | .8325 | .6749 | .6088 | .7716 | .6806 |
| PacBio 4X | .5542 | .8656 | .6757 | .6487 | .9236 | .7621 | .5391 | .8606 | .6630 | .4627 | .8532 | .6000 |
| PacBio 30X | .6321 | .9048 | .7442 | .7541 | .9475 | .8398 | .6153 | .8973 | .7300 | .5906 | .8789 | .7065 |
| high depth | | | | | | | | | | | | |

### Performance by SV Type

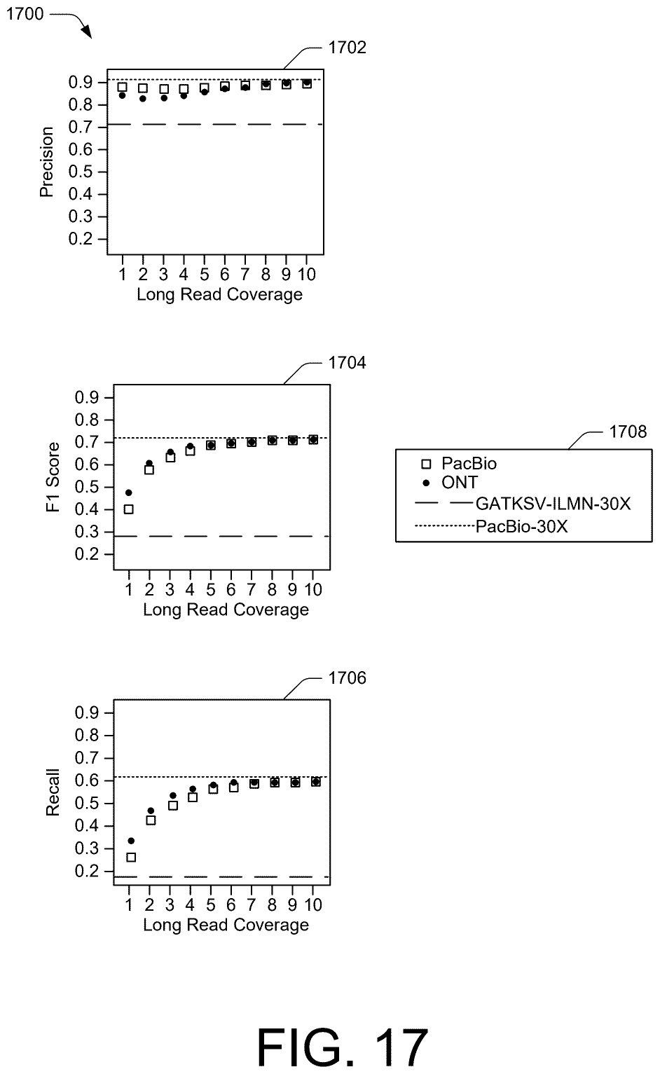

FIG. 17 shows an example 1700 of insertion structural variant discovery with long reads versus short reads. The example 1700 includes a first graph 1702 of precision (vertical axis) versus long read coverage (horizontal axis), a second graph 1704 of F1 score (vertical axis) versus long read coverage (horizontal axis), and a third graph 1706 of recall (vertical axis) versus long read coverage (horizontal axis). A legend 1708 indicates that long read data obtained via PacBio is indicated by white (or open-filled) squares, long read data obtained via ONT is indicated by black circles, the GATK-SV callset is indicated by long-dashed lines, and 30× long-read performance is indicated by short-dashed lines.

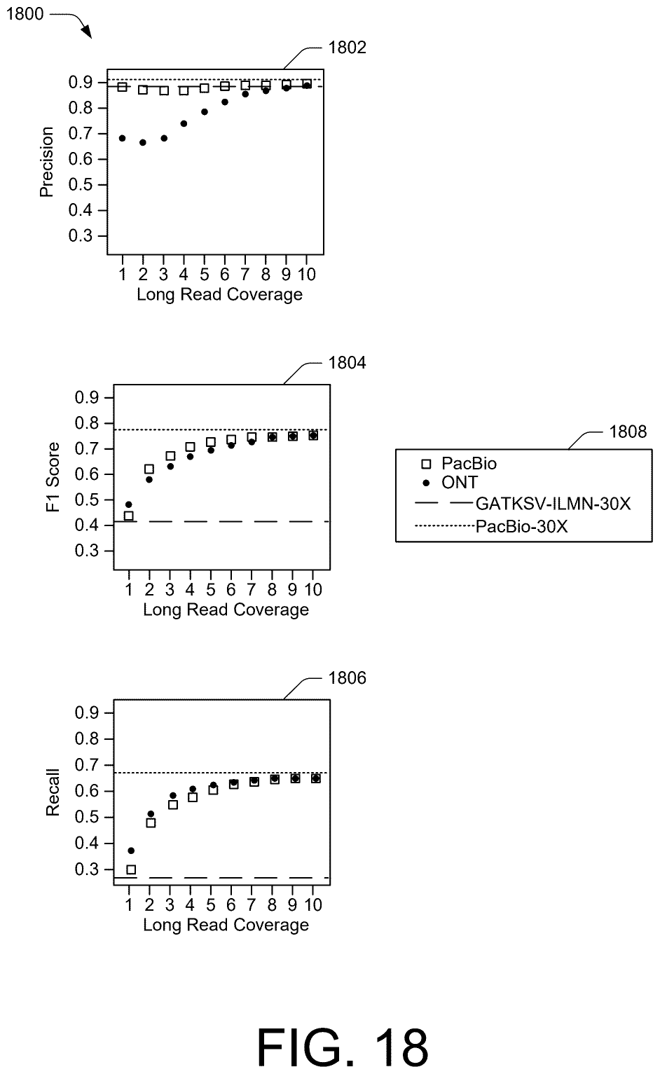

FIG. 18 shows an example 1800 of deletion structural variant discovery with long reads versus short reads. The example 1800 includes a first graph 1802 of precision (vertical axis) versus long read coverage (horizontal axis), a second graph 1804 of F1 score (vertical axis) versus long read coverage (horizontal axis), and a third graph 1806 of recall (vertical axis) versus long read coverage (horizontal axis). A legend 1808 indicates that long read data obtained via PacBio is indicated by white (or open-filled) squares, long read data obtained via ONT is indicated by black circles, the GATK-SV callset is indicated by long-dashed lines, and 30× long-read performance is indicated by short-dashed lines.

When breaking down the results by SV type, a difference in performance improvement for insertions (FIG. 17) versus deletions (FIG. 18) is observed. Blend-seq results in large gains in recall (the third graph 1706 and the third graph 1806) for both categories, but for deletions, the gain is smaller for precision (the first graph 1802) since short reads achieve relatively high precision in this category. This is not surprising, given that short reads can gather accurate depth evidence for large deletions but struggle to assemble large inserted sequences. Note that even if deletions are further restricted to regions where short reads are expected to be most competitive, i.e., the regions that are easiest for short reads to map (“Easy Regions”), it is still found that four PacBio long read layers substantially outperform short-read performance in both precision and recall.

The results were broken down by SV length to determine whether performance varies between relatively short (100-250 bp) events and longer (2.5 k-10 k) events.

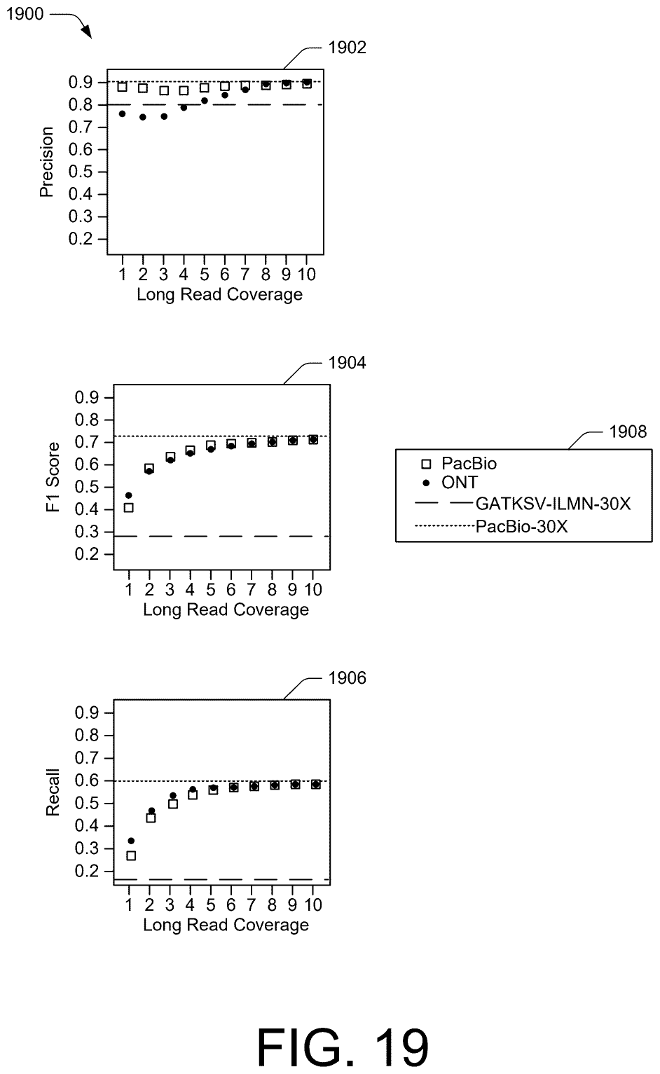

FIG. 19 shows an example 1900 of structural variant discovery for structural variants of length 100-250 base pairs with long reads versus short reads. The example 1900 includes a first graph 1902 of precision (vertical axis) versus long read coverage (horizontal axis), a second graph 1904 of F1 score (vertical axis) versus long read coverage (horizontal axis), and a third graph 1906 of recall (vertical axis) versus long read coverage (horizontal axis). A legend 1908 indicates that long read data obtained via PacBio is indicated by white (or open-filled) squares, long read data obtained via ONT is indicated by black circles, the GATK-SV callset is indicated by long-dashed lines, and 30× long-read performance is indicated by short-dashed lines.

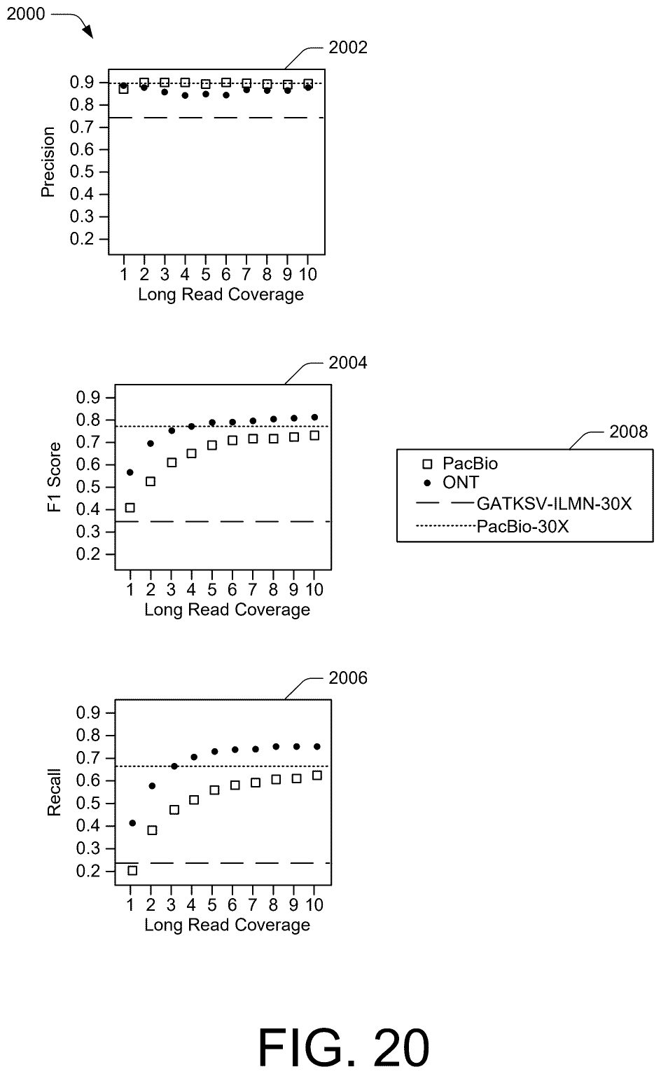

FIG. 20 shows an example 2000 of structural variant discovery for structural variants of length 2500-10000 base pairs with long reads versus short reads. The example 2000 includes a first graph 2002 of precision (vertical axis) versus long read coverage (horizontal axis), a second graph 2004 of F1 score (vertical axis) versus long read coverage (horizontal axis), and a third graph 2006 of recall (vertical axis) versus long read coverage (horizontal axis). A legend 2008 indicates that long read data obtained via PacBio is indicated by white (or open-filled) squares, long read data obtained via ONT is indicated by black circles, the GATK-SV callset is indicated by long-dashed lines, and 30× long-read performance is indicated by short-dashed lines.

The expectation was that a difference between PacBio and ONT performance would be observed. While ONT reads are longer (mean 50.7 kb with standard deviation 66.9 kb), PacBio reads (mean 15.6 kb with standard deviation 3.9 kb) have higher per-base accuracy, so ONT was expected to do better in longer events and PacBio in shorter events. This is indeed the case, as shown in Table 4. The PacBio reads show better precision for smaller events (the first graph 1902) even at extremely low coverage, and the ONT recall outperforms the PacBio reads for larger events (the third graph 2006). In both cases, long reads outperform the short read control.

TABLE 4
SV Discovery by Event Size

| | 100-250 | | | 2.5k-10.0k | | |
|---|---|---|---|---|---|---|
| | Recall | Precision | F1 Score | Recall | Precision | F1 Score |
| Illumina 30x (control) | .1582 | .8083 | .2647 | .2268 | .7378 | .3470 |
| ONT 1x | .3274 | .7620 | .4580 | .4050 | .8740 | .5536 |
| PacBio 1x | .2606 | .8837 | .4025 | .1935 | .8611 | .3160 |
| ONT 4x | .5503 | .7933 | .6499 | .6922 | .8275 | .7538 |
| PacBio 4x | .5337 | .8672 | .6607 | .5050 | .8783 | .6412 |
| PacBio 30x (high depth) | .5970 | .9093 | .7208 | .6535 | .8784 | .7494 |

### Genotype-Aware Structural Variant Discovery

In this subsection, the setting is considered where structural variants are not only found, but also genotyped correctly. This is not performed for all applications, but in diagnostic scenarios, for instance, knowing the difference between a heterozygous versus a homozygous variant may have substantial implications on a potential loss of function or other disruptions in the transcriptome. For this setting, a method is developed in which a sample-specific graph reference is created from the low-coverage long reads. Then, the short reads are mapped to this reference, and variant calling is performed on the graph. Results are presented across the entire genome, showing modest improvements with respect to using short or long reads alone. Results are also presented for easier-to-map regions, where the improvement is more substantial because the short reads can provide greater assistance there.

### Approach and Data

While it was shown that SV recall and precision (agnostic of genotype) could be substantially improved in the previous section, here the exploration is whether genotype-aware SV calling performance can be improved by integrating short-read information. The approach was to create a “personalized” graph reference for the sample using the low-coverage long-read alignments, then call SVs from short reads aligned to the graph. In this way, the aims were to improve precision by avoiding calls that did not have enough short-read support and to improve genotyping accuracy by taking advantage of the higher-depth short reads. In the previous subsection, 4× long reads were found to be a reasonable tradeoff between coverage and SV discovery performance. Thus, 4× was chosen as a fixed setting of long-read coverage in this subsection. The short-read coverage was then varied from 10 to 60× to explore the marginal benefit from additional layers of short reads.

It is noted that although personalized graph references for short-read mapping are under active development in the bioinformatics research space, they are typically built by subsetting a large cohort pangenome to the parts that are most similar to the short reads for the sample at hand. As such, they are unlikely to contain rare SVs carried by the sample. In the techniques described herein, on the other hand, the personalized reference is built from sample-specific low-coverage long reads.

For each long-read technology (PacBio or ONT), the vg toolkit was used to create the graph reference from the long-read alignments. This involved taking all structural variants (INDELs with length ≥50 bp) from the CIGAR encoding of the alignments and writing them to a variant call format (VCF) file. This VCF file was used as the input to vg to create the graph. The short reads were then mapped to the graph. Next, vg was used to call variants from these alignments, and the resulting VCF went through a postprocessing step described in the Methods section before comparing to the NIST-Q100 HG002 SV truth set with Truvari. Truvari was run with all parameters set to defaults except for sequence comparisons, which were turned off. This process generated true positive (TP), false positive (FP), and false negative (FN) labels, from which the precision, recall, and F1 scores were calculated.

Truvari compares events based on proximity and size. The tool allows some amount of flexibility to account for both consensus calls made from slightly different alignments as well as for reference ambiguity in repetitive regions. As opposed to traditional short-variant statistics, by default, Truvari will label a “true positive” event based solely on the comparison of alleles between variants without genotype information taken into account, which was the output used in the previous subsection. The tool also reports TP matches with genotype information forced to match. This allows for six different performance statistics (precision, recall, and F1 for both genotype-agnostic and genotype-aware) to be calculated depending on whether the match should be forced to match genotype in addition to the site-level match. To clarify that the genotype-aware values are being referred to in these figures and tables, they are labeled as, e.g., “GT-Precision” instead of “Precision.”

### Performance Across the Entire Genome

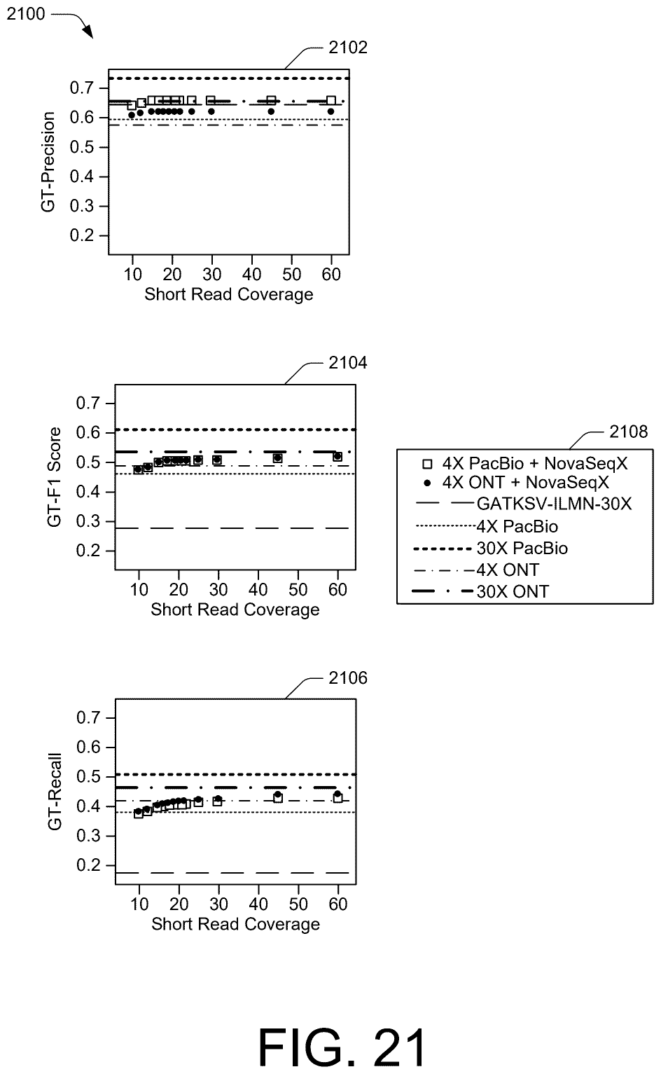

FIG. 21 shows an example 2100 of genotype-aware structural variant discovery with long reads alone, short reads alone, or a hybrid method. The example 2100 includes a first graph 2102 of precision (vertical axis) versus short read coverage (horizontal axis), a second graph 2104 of F1 score (vertical axis) versus short read coverage (horizontal axis), and a third graph 2106 of recall (vertical axis) versus short read coverage (horizontal axis). A legend 2108 indicates that data obtained using 4× PacBio and NovaSeq (with varying short read coverage) is indicated by white (or open-filled) squares, data obtained via 4×ONT and NovaSeq (with varying short read coverage) is indicated by black circles, the GATK-SV callset is indicated by long-dashed lines, 4× PacBio coverage is indicated by thinner short-dashed lines, 30×PacBio is indicated by thicker short-dashed lines, 4×ONT coverage is indicated by thinner dot-dashed lines, and 30×ONT coverage is indicated by thicker dot-dashed lines.

For the genotype-aware setting, better performance is seen with the blend-seq approach across all categories. This is expected, since the short reads have much higher depth than the long reads. While the long reads lend their superior mapping capabilities and lengths to better SV haplotype discovery, the short reads can be used to help resolve the genotypes of those haplotypes. Detailed values are shown in Table 5.

TABLE 5
Genotyping SV Performance by Region

| | Whole Genome | | | Easy Regions | | | Hard Regions | | |
|---|---|---|---|---|---|---|---|---|---|
| | GT- | GT- | GT-F1 | GT- | GT- | GT-F1 | GT- | GT- | GT-F1 |
| | Recall | Precision | Score | Recall | Precision | Score | Recall | Precision | Score |
| IL 30X control | .1741 | .6411 | .2738 | .3458 | .7382 | .4710 | .1409 | .6076 | .2288 |
| ONT 4X LR-only | .4206 | .5773 | .4866 | .4752 | .5390 | .5051 | .4069 | .5964 | .4837 |
| PacBio 4X LR-only | .3815 | .5959 | .4652 | .4314 | .6155 | .5073 | .3672 | .5858 | .4514 |
| blend-seq ONT 4X + | .3868 | .6068 | .4724 | .4738 | .7244 | .5729 | .3693 | .5908 | .4545 |
| IL 10X | | | | | | | | | |
| blend-seq PacBio | .3753 | .6385 | .4727 | .4531 | .7412 | .5624 | .3557 | .6191 | .4518 |
| 4X + IL 10X | | | | | | | | | |
| blend-seq ONT 4X + | .4314 | .6218 | .5094 | .5153 | .7205 | .6009 | .4147 | .6076 | .4930 |
| IL 30X | | | | | | | | | |
| blend-seq PacBio | .4179 | .6517 | .5092 | .4858 | .7386 | .5861 | .3995 | .6341 | .4902 |
| 4X + IL 30X | | | | | | | | | |
| ONT 30X LR-only | .4601 | .6524 | .5396 | .5389 | .6230 | .5779 | .4454 | .6427 | .5262 |
| PacBio 30X LR- | .5124 | .7334 | .6033 | .5847 | .7349 | .6512 | .4936 | .7198 | .5856 |
| only | | | | | | | | | |

### Performance in Easier Regions

The boost in genotype-aware performance provided by the short reads is greatest where the short reads are best able to map. As in the last subsection, these are referred to as “Easy Regions,” which include the complement of NIST's “AllDifficult” regions. This setting reduces the total 30 k variants in the truth dataset to 11.5 k overlapping or contained within this region. In the genotype-aware setting, the hybrid method outperforms 4× long reads alone in terms of precision, recall, and F1, beginning around 12× of short reads when paired with ONT and 20× of short reads when paired with PacBio. These performance increases continue up to 60× short reads. Between the ONT and PacBio technologies, something of a precision-recall tradeoff is seen: PacBio has the upper hand for precision and ONT for recall in these plots, as would be expected from the discussion in the previous subsection. Detailed numbers are supplied in Table 5.

The genotype-aware performance can be broken down further in this region by SV type. As expected, the biggest gains are in deletions, since this is where short reads can contribute the most. The values for all statistics for deletions are recorded in Table 6. The performance boost is more modest but still visible for insertions, as seen in Table 7.

TABLE 6
Genotyping SV Deletions Performance by Region

| | Whole Genome | | | Easy Regions | | | Hard Regions | | |
|---|---|---|---|---|---|---|---|---|---|
| | GT- | GT- | GT-F1 | GT- | GT- | GT-F1 | GT- | GT- | GT-F1 |
| | Recall | Precision | Score | Recall | Precision | Score | Recall | Precision | Score |
| IL 30X control | .2292 | .7436 | .3503 | .7688 | .8289 | .7977 | .2054 | .7233 | .3199 |
| ONT 4X LR-only | .5003 | .5961 | .5440 | .7476 | .5246 | .6165 | .4928 | .6507 | .5608 |
| PacBio 4X LR-only | .4553 | .6674 | .5413 | .6924 | .7699 | .7291 | .4463 | .6666 | .5347 |
| blend-seq ONT 4X + | .4707 | .6692 | .5527 | .8109 | .8928 | .8499 | .4581 | .6590 | .5405 |
| IL 10X | | | | | | | | | |
| blend-seq PacBio | .4651 | .7102 | .5621 | .7707 | .9332 | .8442 | .4529 | .6987 | .5496 |
| 4X + IL 10X | | | | | | | | | |
| blend-seq ONT 4X + | .5093 | .7051 | .5914 | .8217 | .8971 | .8577 | .4976 | .6963 | .5804 |
| IL 30X | | | | | | | | | |
| blend-seq PacBio | .4985 | .7431 | .5967 | .7777 | .9372 | .8500 | .4870 | .7332 | .5852 |
| 4X + IL 30X | | | | | | | | | |
| ONT 30X LR-only | .4942 | .6797 | .5723 | .6569 | .6466 | .6517 | .4848 | .6759 | .5646 |
| PacBio 30X LR- | .6092 | .8235 | .7003 | .9016 | .9138 | .9077 | .5972 | .8167 | .6899 |
| only | | | | | | | | | |

TABLE 7
Genotyping SV Insertions Performance by Region

| | Whole Genome | | | Easy Regions | | | Hard Regions | | |
|---|---|---|---|---|---|---|---|---|---|
| | GT- | GT- | GT-F1 | GT- | GT- | GT-F1 | GT- | GT- | GT-F1 |
| | Recall | Precision | Score | Recall | Precision | Score | Recall | Precision | Score |
| Illumina 30x (control) | .1406 | .5640 | .2250 | .2257 | .6733 | .3381 | .1003 | .5023 | .1672 |
| ONT 4x (LR-only) | .5003 | .5961 | .5440 | .7476 | .5246 | .6165 | .4928 | .6507 | .5608 |
| PacBio 4x (LR-only) | .3366 | .5477 | .4170 | .3573 | .5549 | .4347 | .3172 | .5289 | .3966 |
| blend-seq ONT 4x + | .3357 | .5621 | .4203 | .3781 | .6496 | .4779 | .3132 | .5392 | .3963 |
| Illumina 10x | | | | | | | | | |
| blend-seq PacBio 4x + | .3206 | .5863 | .4145 | .3629 | .6591 | .4681 | .2944 | .5574 | .3853 |
| Illumina 10x | | | | | | | | | |
| blend-seq ONT 4x + | .3841 | .5677 | .4582 | .4283 | .6506 | .5165 | .3624 | .5471 | .4360 |
| Illumina 30x | | | | | | | | | |
| blend-seq PacBio 4x + | .3688 | .5918 | .4545 | .4029 | .6616 | .5008 | .3444 | .5658 | .4281 |
| Illumina 30x | | | | | | | | | |
| ONT 30x (LR-only) | .4942 | .6797 | .5723 | .6569 | .6466 | .6517 | .4848 | .6759 | .5646 |
| PacBio 30x (LR-only) | .4535 | .6732 | .5419 | .4946 | .6669 | .5680 | .4283 | .6518 | .5169 |

### Discussion

Blend-seq, by combining or selecting from standard-coverage short reads and low-coverage long reads, can provide substantial gains in SNP precision and recall in structural variant (SV) discovery and in genotyping structural variants, as demonstrated with specific choices of datasets, pipelines, and settings.

In terms of genomic datasets, all results were presented on a single genome, HG002, for ease of accessing high-quality truth data. A proxy for ground truth could be created using high-quality assemblies like those in the Human Pangenome Reference Consortium or the Human Genome Structural Variation Consortium, then leveraging assembly-based variant callers like Dipcall or hapdiff. In a similar vein, by using a complete reference genome, like the Telomere-to-Telomere (T2T) reference, mapping quality may be further improved, and better variant calls made, using blend-seq techniques.

Further, the examples provided herein in the Example Application represent one set of choices. Several other long-read mappers exist, as well as several other SV callers and genotypers, both for long and for short reads, each with parameters that could be tuned for particular applications. In other examples, multiple long-read SV callers (including possibly some assembly-based callers) could be run and their output merged and filtered using features that come from both long- and from short-read alignments. In addition, bespoke methods for specific hybrid regimes could be developed, both for SV and for SNP/INDEL calling.

The sequencing technologies investigated herein represent some of the most popular options but are only a subsample of those that are available. By way of example, any mix of technologies may provide benefits of similar magnitude to variant discovery, and different error mode combinations may provide opportunities for optimizing mixes for specific applications. In addition to the PacBio-Illumina and ONT-Illumina combinations described herein in the Example Application, hybrid SV genotyping performance using Ultima short reads was also investigated. The resulting performance is almost identical to hybrid Illumina.

For short variants, the performance of SNPs alone (versus INDELs) was emphasized for two reasons. The first reason is that the long reads used added extra short INDELs in their alignments, which led to a greater rate of false positives than the short reads alone and reduced overall precision. Despite this, a similar boost in INDEL recall was observed, and it is contemplated that with additional tuning of available filtering models (either for the alignments or for the variants), the boost in recall may be retained without sacrificing precision, similar to the SNPs. The second reason is that tabulating read support for INDELs is a more subtle problem than for SNPs, as representation differences in alignments have to be resolved, especially in repetitive regions. This also may be handled by custom methods for normalizing the alignments when calculating hybrid read support.

Also in the context of short variants, the DRAGEN version used in this Example Application (v3.7.8) was chosen to match the version used by the All of Us Research Program, a large biobank funded by the U.S. government. It should be noted that recent work shows improved performance of DRAGEN in later versions. However, no versions past v3.7.8 have an open-source implementation, while the variant caller used should be functionally equivalent to DRAGEN-GATK, which also influenced the decision to use an earlier version.

Because phasing information was not readily available for the hybrid data, tools like truvari refine or vcfdist could not be used to better compare the resulting haplotypes to the truth. This resulted in recall being lower than what was previously reported, which did have phasing information and thus performed this processing step. To ensure this was the source of the discrepancy, the 30× PacBio sample was phased using HiPhase and truvari refine was run. This resulted in a 95% F1 score, which is indeed comparable to prior work. This evaluation may be adapted to blend-seq settings by using a long-read phasing tool compatible with multiple sequencing technologies.

In this way, using blend-seq to combine (or select from) short and long read technologies leads to substantial improvements in variant calling performance in multiple scenarios. In particular, augmenting traditional short-read coverage (30×) with low-coverage long reads (4×) yields benefits that rival a high-coverage long-read pipeline. SNP recall and precision exceed short reads even at very high coverage (60×), structural variant (SV) recall and precision far exceed short-read capabilities and approach full long-read performance, and substantial improvements in genotype-aware SV calling precision and recall over short-read pipelines are achieved.

These gains are a result of the complementary capabilities of short and long read technologies. While short reads can provide many layers of high-accuracy coverage at low cost, they struggle with regions that are ambiguous to map with such a short size. Long reads, on the other hand, excel at mapping to these difficult regions and can also provide high accuracy per-base information, but at a much greater cost per layer. The greatest gains from this approach were observed where this complementarity is strongest. For example, long reads help with difficult-to-map regions for SNPs, long reads better capture long insertions and deletions (structural variants), and the many layers of short reads help provide genotyping signal for variants discovered by long reads. By combining or selecting from these technologies as shown herein in the Example Application, the best of both can be achieved with only a few layers of long reads augmenting a traditional short-read run. It is to be appreciated that the techniques described herein are adaptable to different variant calling pipelines in different ratios and using different methods of combination without departing from the spirit or scope of the describe disclosure.

### Methods

In this section, the pipelines and settings used for the various analyses of the Example Application are described in detail.

### Short Variant Calling

Blend-seq short variant calls were generated by combining preprocessed long reads with the Illumina short reads to create a hybrid Binary Alignment Map (BAM). This BAM was used as input to DRAGEN v3.7.8, with the version selected to match what is used for the All of Us Research Program. The tool was designed for Illumina short reads alone. Therefore, several preprocessing steps on the long reads were used to enable DRAGEN to run. The long read BAMs were first downsampled to 4× coverage with samtools to simulate a lower coverage long read product. Then, the following processing steps were performed:
1. In order to remove large scale deletion information which cannot be processed by DRAGEN which is designed for short INDELs alone, large deletions in the long read alignments were converted to use “N” in their CIGAR strings rather than “D” via a custom pysam script.
2. GATK's SplitNCigarReads was used to split alignments at these large deletions to provide cleanly aligned reads as a technical intermediate to feed into the following custom tool.
3. To simulate a short read structure from the long reads, a custom chop-reads program was written to chop the alignments evenly at 151 read bases and update the CIGAR strings accordingly. This allows DRAGEN to see reads of equal length regardless of which technology generated the read, while still keeping the alignment information generated from the original long reads.
4. To generate the final hybrid BAM, samtools was used to sort and then merge these reads with the DRAGMAP aligned short reads. The header was also updated to just have one read group via samtools reheader in order to work around a limitation in the DRAGEN version used.
5. Chopped long reads with CIGAR string equal to a pure insertion (i.e., “1511”) were removed, since these are not useful for short INDEL calling and caused errors in the DRAGEN pipeline.
6. Then, samtools was used to index these hybrid BAMs, and they were run through the full DRAGEN variant calling tool.

For comparison with the hybrid samples, control samples for just long and just short reads were processed using standard SNP and short INDEL calling pipelines for long and short reads, respectively. The long reads were aligned using minimap2, and the short reads were aligned using DRAGMAP as a part of the DRAGEN variant calling process, using the GRCh38 human reference in both cases. The long reads (PacBio and ONT) were run through Deep Variant (using v1.3 for PacBio and v1.6 for ONT), while the short reads (Illumina) were run through DRAGEN v3.7.8.

After obtaining the hybrid DRAGEN Variant Call Format files (VCFs) at each desired mixture of coverages, benchmarking was performed against the NIST HG002 v4.2.1 standard Genomes in a Bottle (GIAB) truth set. A pipeline wrapping RTG's vcfeval tool was used, which resolves haplotypes underlying variant representations to ensure accurate comparisons. From this, true positive (TP), false positive (FP), and false negative (FN) labels were obtained from which precision, recall, and F1 statistics are derived, including over different subsets of the genome. These BED file regions (“LowMappability All,” and others used in this paper) were also curated by NIST for their GIAB release.

To create the pie charts demonstrating the breakdown of types of short read evidence (or lacking) among the variants uniquely picked up by the hybrid method, the set of SNPs labeled with FN from vcfeval comparing the pure short read VCF against the NIST truth was taken and intersected with those labeled TP from vcfeval comparing the hybrid VCF against the NIST truth. Then this was restricted to those contained in NIST's low mappability regions, where the largest boost in performance was observed. Each SNP was given a unique label in the following list with higher labels taking precedence, depending on how the evidence in the pure short read BAM looked:
A. Zero Reads: zero depth (DP) at the site;
B. Zero Alt-Supporting Reads: zero short reads supporting the SNP in their alignment;
C. Only MAPQ Zero Reads: all reads above site have mapping quality (MQ) 0;
D. Only MAPQ Zero Alt-Supporting Reads: all reads supporting SNP in alignment have MQ 0;
E. Low Depth: total DP is less than 2;
F. Low Alt-Supporting Depth: total DP among reads supporting SNP is less than 2;
G. Low MAPQ Reads: the average of the mean MQ reference-supporting reads and the mean MQ of the SNP-supporting reads is less than or equal to 20;
H. Low BaseQ Alt-Supporting Reads: the average base qualities of reads supporting the SNP is less than 20;
I. Other: all other SNPs.

This provides a somewhat sorted categorization from categories that are harder to call with short reads alone to those that are easier to call with short reads SNPs.

Structural Variant Discovery with Long Reads

In order to measure structural variant (SV) discovery at various levels of coverage, each set of long reads (PacBio and ONT) were downsampled from 1-10×, and then run through sniffles v2.2 to call SVs. Parameters were chosen to be more sensitive at lower coverage. In particular, it was run with settings: --minsupport 1 --qc-output-all --qc-coverage 1 --long-dup-coverage 1 --detect-large-ins True.

A custom script was then used to clean the data (CleanSVs.wdl), which splits multiallelic sites, recomputes the SVLEN annotation (SV Length) to standardized positive values and fill in missing values, strips any filters to maximize recall, and removes events smaller than 50 bp, which fall under the short INDEL category (see above). Following this step, the cleaned VCF was run through a pipeline wrapping truvari v.4.0.0, setting dup-to-ins and passonly to be true, and using petseq equal to 0 in order to have a more equal comparison with the short read control, which do not report base pair resolution of differences in large insertions. The SV VCF was also restricted to events with AC at least 1 using bcftools view --min-ac 1, so reference calls were removed before running truvari. These were compared to the NIST Q100 v1.0 SV truth set for HG002, which is part of the T2T-Q100 project, an initiative attempting to create perfect telomere-to-telomere assemblies and polishing them with new tools like in the Telomere-to-Telomere project. The truth VCF was also restricted to events of size at least 50 bp, and multiallelic sites were split in the same way that the comparison VCFs were cleaned, except filters were kept in the truth.

Only events with at least 25% overlap with NIST's truth high confidence BED file (labeled stvar.benchmark.bed) were considered for benchmarking statistics. After obtaining TP, FP, and FN labels in this way, precision, recall, and F1 statistics were derived overall, and also when restricting to events with positive percent overlap in particular regions of interest. When considering overlaps with specific regions, the convention that insertions should have reference length given by the length of the inserted sequence was followed in order to avoid nearby misses due to ambiguity in the exact starting base, particularly when considering events discovered by short reads. As a control, the performance was compared to a callset curated from GATK-SV calls on an Illumina 30×BAM that was processed with the same cleaning and benchmarking pipeline.

### Hybrid Genotyping of SVs

Starting with 4× long reads (PacBio and ONT) aligned to the GRCh38 human reference, a sample-specific graph reference was created to map the short reads against using vg v1.54.0. An overview of the pipeline implementation is as follows:
1. “Trivial SV calls” were created using long read alignments at places where the CIGAR string has an insertion or deletion exceeding 50 bp using a custom tool. This created a VCF of naive SV candidates from the long read alignments themselves and was deduplicated using bcftools norm-d.
2. A graph reference was obtained by augmenting the linear GRCh38 human reference using these trivial SV calls using vg autoindex-workflow giraffe.
3. The short read FASTQs were split using seqkit split2 to parallelize the short read alignments.
4. Each short read batch was mapped to the sample-specific graph reference via vg giraffe with default parameters.
5. The resulting Graph Alignment Maps (GAMs) were collected, and structural variants were called using vg pack and vg call, which is the mode for calling novel SVs rather than re-genotyping a specified set.

The output was then run through the same cleaning and benchmarking process as described in the last section. The resulting performance statistics were plotted relative to their short read coverage and compared to the pure short read and pure long read controls, the latter of which were obtained as in the last section.

### Data Sources

For each technology, a high coverage set of reads was downsampled to the approximately desired coverage using samtools view-s.

For Illumina reads, two replicates of HG002 were sequenced on a NovaSeq X machine and merged into a 68×BAM. It was aligned using DRAGMAP v3.7.8. This was first downsampled to 60× and then to a desired coverage.

For PacBio reads, 30× HG002 run on a Revio machine sequenced for internal validation studies was used. It was aligned using pbmm2 with the flags --preset CCS --sample NA24385_1 --strip --sort --unmapped.

For ONT, a set of reads totaled around 14× was used, and then downsampled to a desired coverage.

Some experiments with Ultima reads were performed, starting around 60× coverage.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A system comprising:
a coverage modulation engine to select a short read sequencing coverage and a long read sequencing coverage for a nucleic acid sequencing event based on a sequencing target of the nucleic acid sequencing event and at least one constraint;
a sequencing protocol generator to determine a short read sequencing protocol based on the selected short read sequencing coverage and determine a long read sequencing protocol based on the selected long read sequencing coverage; and
an integrated variant calling module to output a variant call based on short read sequencing data generated via the short read sequencing protocol and long read sequencing data generated via the long read sequencing protocol.

2. The system of claim 1, wherein the coverage modulation engine generates and stores relationships between the at least one constraint and sequencing coverages for a plurality of different combinations of short read and long read sequencing coverages and based on reference short read sequencing data and reference long read sequencing data obtained for a reference sample.

3. The system of claim 2, wherein the coverage modulation engine selects the short read sequencing coverage and the long read sequencing coverage based on the relationships and based on a multi-objective algorithm.

4. The system of claim 1, further comprising an alignment module to:
align the short read sequencing data and the long read sequencing data with respect to one or more reference sequences and with respect to each other; and
generate a sequence of a sample evaluated by the nucleic acid sequencing event based on an order of nucleotide bases specified by the aligned short read sequencing data and the aligned long read sequencing data.

5. The system of claim 4, wherein the variant call output by the integrated variant calling module is further based on one or more differences between the sequence of the sample and the one or more reference sequences.

6. The system of claim 1, wherein the at least one constraint comprises one or more of a sensitivity for identifying at least one type of variant in at least a portion of a reference sequence, a precision for identifying the at least one type of variant in at least the portion of the reference sequence, and an accuracy of identifying nucleotide bases in the reference sequence.

7. The system of claim 1, wherein a sensitivity of the variant call and a precision of the variant call are increased compared to performing the variant call using the short read sequencing data without the long read sequencing data and compared to performing the variant call using the long read sequencing data without the short read sequencing data.

8. The system of claim 1, wherein the variant call identifies at least one of a single nucleotide polymorphism, an insertion, a deletion, a copy number variant, a duplication, an inversion, a replacement, or a translocation in a sample sequenced by the nucleic acid sequencing event relative to a reference sequence, and wherein the variant call further includes variant phasing information.

9. A method comprising:
receiving a selection of at least one target of a genomic sequencing event and at least one constraint of the genomic sequencing event;
selecting a short read sequencing coverage and a long read sequencing coverage for the genomic sequencing event based on the at least one target and the at least one constraint;
generating a short read sequencing protocol based on the short read sequencing coverage; and
generating a long read sequencing protocol based on the long read sequencing coverage.

10. The method of claim 9, wherein the at least one constraint comprises a performance constraint defining a targeted performance metric of the genomic sequencing event and a resource constraint of the genomic sequencing event.

11. The method of claim 10, wherein the targeted performance metric is one or more performance metrics selected from a group comprising an accuracy metric, a sensitivity metric, and a precision metric.

12. The method of claim 9, wherein selecting the short read sequencing coverage and the long read sequencing coverage for the genomic sequencing event based on the at least one target and the at least one constraint comprises:
referencing a relationship library storing a plurality of relationships between the at least one constraint and sequencing coverages for a plurality of different combinations of short read and long read sequencing coverages obtained for a reference sample and a plurality of different targets.

13. The method of claim 9, further comprising sequencing a nucleic acid sample using the long read sequencing protocol and the short read sequencing protocol.

14. A method comprising:
receiving short read sequencing data generated for a biological sample via a short read sequencing protocol having a first sequencing coverage, the short read sequencing data comprising sequences of nucleotide bases for a plurality of short nucleotide fragments;
receiving long read sequencing data generated for the biological sample via a long read sequencing protocol having a second sequencing coverage, the long read sequencing data comprising sequences of the nucleotide bases for a plurality of long nucleotide fragments, wherein the first sequencing coverage and the second sequencing coverage are selected based on at least one constraint;
generating at least one alignment of the short read sequencing data and the long read sequencing data with a reference genome; and
outputting a variant call based on the at least one alignment relative to the reference genome.

15. The method of claim 14, wherein the first sequencing coverage and the second sequencing coverage are selected by a coverage modulation engine using a multi-objective algorithm and based on the at least one constraint, and wherein the at least one constraint comprises one or more of a sensitivity for identifying at least one type of variant in at least a portion of a genomic sequence, a precision for identifying the at least one type of variant in at least the portion of the genomic sequence, an accuracy of identifying the nucleotide bases in at least the portion of the genomic sequence, and a resource constraint for generating the genomic sequence of the biological sample.

16. The method of claim 15, wherein the first sequencing coverage and the second sequencing coverage are further selected by the coverage modulation engine based on a sequencing target.

17. The method of claim 14, wherein outputting the variant call based on the at least one alignment relative to the reference genome comprises:
after aligning the long read sequencing data, fragmenting the long read sequencing data into aligned synthetic short reads;
generating a hybrid alignment by combining the aligned synthetic short reads with a short read alignment of the short read sequencing data; and
outputting the variant call based on the hybrid alignment.

18. The method of claim 14, wherein generating the at least one alignment of the short read sequencing data and the long read sequencing data with the reference genome comprises:
generating an augmented reference genome by mapping the long read sequencing data to the reference genome; and
mapping the short read sequencing data to the augmented reference genome.

19. The method of claim 14, wherein the first sequencing coverage comprises an average number of reads at a specific location in a genome that is less than thirty, and wherein the second sequencing coverage is less than the first sequencing coverage.

20. The method of claim 14, further comprising:
identifying phasing of variants output in the variant call, the phasing indicating which of the variants occur on a same copy of a chromosome.
